(19) United States
(12) Patent Application Publication
(10) Pub. No.: US 2022/0325261 A1
(43) Pub. Date: Oct. 13, 2022

(54) PTPRS AND PROTEOGLYCANS IN AUTOIMMUNE DISEASE

(71) Applicant: La Jolla Institute for Immunology, La Jolla, CA (US)

(72) Inventors: Nunzio Bottini, La Jolla, CA (US); Stephanie Stanford, La Jolla, CA (US)

(21) Appl. No.: 17/848,241

(22) Filed: Jun. 23, 2022

Related U.S. Application Data
(63) Continuation of application No. 14/416,839, filed on Jan. 23, 2015.
(60) Provisional application No. 61/832,688, filed on Jun. 7, 2013.
(60) Provisional application No. 61/675,036, filed on Jul. 24, 2012.
(60) Provisional application No. 61/674,853, filed on Jul. 23, 2012.

(51) Int. Cl. C12N 9/16; C07K 14/705; A61K 39/395; C07K 16/40; C07K 16/18; G01N 33/68

(52) U.S. Cl. CPC C12N 9/16; C07K 14/705; A61K 39/395; C07K 16/40; C07K 16/18; G01N 33/68; C12Y 301/03048; G01N 2500/02

(57) ABSTRACT

Provided herein, inter alia, are PTPRS de-clustering agents and compositions and kits comprising the agents. Provided are methods of modulating extracellular matrix or decreasing fibroblast activity in a subject. Also provided are methods of treating subjects with or at risk of developing extracellular matrix diseases, fibroblast-mediated diseases, or autoimmune diseases.

### CROSS-REFERENCES TO RELATED APPLICATIONS

This application claims priority to U.S. Provisional Application No. 61/674,853, filed Jul. 23, 2012; U.S. Provisional Application No. 61/675,036, filed Jul. 24, 2012; and U.S. Provisional Application No. 61/832,688, filed Jun. 7, 2013. These applications are incorporated by reference herein in their entireties.

### REFERENCE TO A SEQUENCE LISTING, A TABLE OR A COMPUTER PROGRAM LISTING APPENDIX SUBMITTED AS AN ASCII TEXT FILE

Sequence Listing written in file 93138-880314_ST25.TXT, created on Jul. 23, 2013, 63,541 bytes, machine format IBM-PC, MS Windows operating system, is hereby incorporated by reference.

### BACKGROUND OF THE INVENTION

Approximately 5 to 8% of people in the United States suffer from an autoimmune disease. Researchers have identified more than 80 different autoimmune diseases and suspect that many more diseases may have an autoimmune component. Rheumatoid arthritis (RA) alone afflicts roughly 2.5 million people in the United States RA affects the joints and bones but may also affect different organs and biological systems.

Fibroblast-like synoviocytes (FLS) are key players in mediating inflammation and joint destruction in rheumatoid arthritis (RA). There is an increased level of attention to this cell type as the possible target of a new generation of anti-RA therapies, which would be used in combination with immunomodulators to help control disease without increasing immune-suppression. The behavior of FLS is controlled by multiple interconnected signal transduction pathways. Several of these pathways involve reversible phosphorylation of proteins on tyrosine residues, which is the result of the balanced action of protein tyrosine kinases (PTKs) and phosphatases (PTPs). PTKs are mediators of FLS growth and invasiveness. PTPs act by removing phosphate groups from phosphorylated tyrosine residues on proteins. Receptor protein tyrosine phosphatases (RPTPs or PTPRs) are PTPs that generally have a variable length extracellular domain followed by a transmembrane region and a C-terminal catalytic cytoplasmic domain. However, little is known regarding the connection between PTPs or PTPRs in relation to FLS. Provided herein are methods and compositions addressing these and other needs in the art.

### BRIEF SUMMARY OF THE INVENTION

Provided herein, inter alia, are compositions and kits comprising a PTPRS de-clustering agent. Optionally, the PTPRS de-clustering agent is a non-enzymatic recombinant protein comprising an amino acid sequence of an extracellular domain of PTPRS or portion thereof.

Also provided are methods of methods of treating an autoimmune disease in a subject, methods of decreasing fibroblast activity in a subject and methods of treating fibroblast-mediated diseases in a subject. The methods include administering to the subject an effective amount of a PTPRS de-clustering agent.

Provided are methods of modulating extracellular matrix in a subject. The methods include administering to the subject an effective amount of a non-enzymatic recombinant protein comprising an amino acid sequence of an extracellular domain of PTPRS or portion thereof.

Provided herein are methods of identifying a candidate PTPRS de-clustering agent. The methods include contacting a test agent with clustered PTPRS proteins and detecting de-clustering of the PTPRS proteins, thereby identifying a candidate PTPRS de-clustering agent. Alternatively, the methods include contacting a test agent with PTPRS and heparan sulfate and determining whether the test agent inhibits binding of the PTPRS to heparan sulfate, inhibition of binding indicating the test agent is a PTPRS de-clustering agent.

### BRIEF DESCRIPTION OF THE DRAWINGS

FIGS. 1A and 1B are graphs depicting expression of PTPRS in fibroblast-like synoviocytes (FLS). FIG. 1A shows expression in mice. FIG. 1B shows expression in humans.

FIGS. 2A, 2B, 2C, 2D, 2E, and 2F are picture and graphs depicting increased bone erosion severity in arthritis for PTPRS −/− mice compared with PTPRS +/+ mice. Mice were injected with 100 μL K/BxN serum intraperitoneally at 8-10 weeks of age (PTPRS−/− received proportionally less serum to reflect their decreased body weight). Ankle thickness was measured with digitals calipers and ankles and wrists were given a clinical arthritis score between 0 (no redness or swelling) and 4 (maximal swelling with digits involved) every two days for 14 days. See FIGS. 2A, 2B, and 2C. After 14 days, mice were euthanized and ankles fixed for histological scoring using H&E (inflammation and bone erosion) and safranin O (cartliage erosion) staining. See FIGS. 2D (inflammation), 2E (cartilage erosion), and 2F (bone erosion) for histology scores.

FIG. 3 is a histogram showing that treatment of mouse FLS (mFLS) with chondroitin sulfate (CS) reduces their invasion through an extracellular matrix. PTPRS−/− mFLS exhibit reduced CS-mediated inhibition of invasion.

FIG. 4 is a graph showing invasiveness of PTPRS−/− mFLS is unaffected by CS at levels approximately 5 times higher than those in the joint.

FIG. 5 is a graph showing that treatment of mFLS with CS reduces their migration. PTPRS−/− mFLS exhibit reduced CS-mediated inhibition of migration.

FIG. 6 are images of gels showing vivo-morpholino mediated knockdown of PTPRS in mouse and human FLS. Left panel: murine MLS: right panel: human FLS.

FIG. 7 is a histogram showing PTPRS knockdown increases migration of human RA FLS and abrogates CS-mediated inhibition of migration.

FIG. 8 are graphs showing mRNA PTPome analysis of RA FLS shows that at least 5 PTPs are highly expressed.

FIGS. 9A and 9B are graphs showing expression of PTPRS mRNA. Expression in FIG. 9A FLS sorted from control mice or mice with K/BxN serum induced acute or chronic arthritis and FIG. 9B mouse sorted macrophages and FLS with PTPRC included as a positive control for expression in macrophages. PTP expression in FLS and macrophages from pooled mice was measured by quantitative polymerase chain reaction. Values are the mean of PTP expression relative to the housekeeping gene GAPDH.

FIGS. 10A and 10B are graphs showing FLS migration. In FIG. 10A, PTPRS WT or KO FLS were allowed to invade through Matrigel-coated transwell chambers. Cells per field were counted after invasion for 24 hours in response to 5% fetal bovine serum (FBS) in the presence of CS. In FIG. 10B, PTPRS WT or KO FLS were allowed to migrate through uncoated transwell chambers. Cells per field were counted after migration for 4 h in response to 5% FBS in the presence of CS with or without chondroitinase ABC treatment. Bars indicate mean +/− standard error of the fold-change of migration relative to that of untreated cells. Data include three independent experiments using three sets of littermate cell lines. (***P<0.001; significance determined using Mann-Whitney test on raw data).

FIGS. 11A and 11B are graphs showing inhibition of RA FLS migration by chondroitin sulfate (CS) is dependent on PTPRS expression. FIG. 11A is a graph of RT-PCR data showing knock-down of PTPRS in RA FLS after incubation with anti-sense oligo (ASO) for 7 days. In FIG. 11B, PTPRS WT or KO FLS were allowed to migrate through uncoated transwell chambers. Cells per field were counted after migration for 4 h in response to 5% fetal bovine serum (FBS) in the presence of CS with or without chondroitinase ABC treatment. Bars indicate mean +/− standard error of the fold-change of migration relative to that of untreated cells. Data include three independent experiments using three sets of littermate cell lines. (***P<0.001; significance determined using Mann-Whitney test on raw data).

FIGS. 12A and 12B are graphs showing displacement of PTPRS from HS is sufficient to inhibit FLS migration and invasion. PTPRS WT or KO FLS were allowed to invade through uncoated transwell chambers. Cells per field were counted after migration for 4 h in response to 5% fetal bovine serum (FBS) in the presence of 20 nM of the Ig1 and Ig2 domains of PTPRS (Ig1+2) alone (FIG. 12A) or in addition to 100 μg/mL CS (FIG. 12B). Bars indicate mean +/− standard error of the fold-change of migration relative to that of untreated cells. Data include three independent experiments using three sets of littermate cell lines. (**P<0.01, ***P<0.001, ****<0.0001; significance determined using Mann-Whitney test on raw data).

FIGS. 13A and 13B are pictures of light micrographs of bovine cartilage explants and a graph. FIG. 13A shows light micrographs stained with Mayer's hematoxylin with and without Ig1+2 treatment. FIG. 13B is a graph showing cell counts with and without Ig1+2 treatment.

FIGS. 14A and 14B are graphs showing beta-catenin and cadherin binding and desphosphorylation. FIG. 14A is a Western blot of total cell lysates or lysates incubated with GST or GST-PTPRS D/A substrate trapping mutant from unstimulated or pervanadate stimulated FLS. * represents GST-PTPRS D1 D/A and ** represents GST tag. FIG. 14B Western blot showing decreased tyrosine phosphorylation of immunopurified beta-catenin in the presence of GST-PTPRS D1 compared to GST alone.

FIG. 15 is a graph showing Ig1 and Ig2 effect in ameliorating arthritis. Clinical Score of BALB/c mice induced with acute arthritis by administration of 100 μL K/BxN serum on Day 0 and treated with vehicle (control) or 500 μg Ig1+2 daily from days 0 to 6. N=3 per treatment. Histopathological analysis of the joint with (lower panel) and without (upper panel) Ig1+2 treatment plus erosion and cartilage damage score.

FIG. 16 is a graph showing treatment of mFLS with recombinant PTPRS Ig1+2 reduces migration.

FIG. 17 is a graph showing PTPRS−/− mFLS are insensitive to recombinant PTPRS Ig1+2 mediated reduced migration.

FIG. 18 is a schematic of proposed PTPRS, chondroitin sulfate and heparin sulfate activity in fibroblast-like synoviocytes in the joint.

### DETAILED DESCRIPTION OF THE INVENTION

The present application relates to the transmembrane PTP commonly referred to as Protein Tyrosine Phosphatase, Receptor Type, S or Sigma (PTPRS). The extracellular domain of PTPRS includes multiple fibronectin type III-like domains and immunoglobulin-like (Ig) domains. The two most external Ig domains (called Ig1 and Ig2) interact with nanomolar affinity with HS—(heparan sulfate) and CS—(chondroitin sulfate) containing PG (proteoglycan). The interaction of PTPRS with HS (which is layered on the cell surface of most cell types) induces a clustered topology of PTPRS, which inhibits the phosphatase action and promotes signaling. On the contrary, CS (enriched in the ECM surrounding neurons and other cells) induces a declustered conformation, which is active and leads to signaling inhibition. Further information regarding PTPRS may be found, for example, in Shen et al., Science 23 Oct. 2009, 592-596; Coles et al., Science 22 April 2011, 484-488.

As demonstrated herein, the extracellular domain of PTPRS binds to proteoglycans in the extracellular matrix. This binding to different proteoglycans results in differences in the intracellular functions of the phosphatase. This PTPRS-mediated mechanism of regulation of intracellular signaling by the extracellular matrix is referred to herein as “the proteoglycan switch”. As demonstrated herein, the proteoglycan switch regulates in a PTPRS-dependent way the adhesion and invasiveness of FLS. Interfering with the proteoglycan switch in vivo leads to decreased severity of arthritis in a mouse model. Thus, as demonstrated herein, PTPRS is a key regulator of RA FLS destructive behavior.

The terms “subject,” “patient,” “individual,” etc. are not intended to be limiting and can be generally interchanged. That is, an individual described as a “patient” does not necessarily have a given disease, but may be merely seeking medical advice.

A “control” or “standard control” refers to a sample, measurement, or value that serves as a reference, usually a known reference, for comparison to a test sample, measurement, or value. For example, a test sample can be taken from a patient suspected of having a given disease (e.g. an autoimmune disease, inflammatory autoimmune disease, cancer, infectious disease, immune disease, or other disease) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. One of skill will recognize that standard controls can be designed for assessment of any number of parameters (e.g. RNA levels, protein levels, specific cell types, specific bodily fluids, specific tissues, synoviocytes, synovial fluid, synovial tissue, fibroblast-like synoviocytes, macrophagelike synoviocytes, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

As used herein, the terms “treat” and “prevent” may refer to any delay in onset, reduction in the frequency or severity of symptoms, amelioration of symptoms, improvement in patient comfort or function (e.g. joint function), decrease in severity of the disease state, etc. The effect of treatment can be compared to an individual or pool of individuals not receiving a given treatment, or to the same patient prior to, or after cessation of, treatment. The term “prevent” generally refers to a decrease in the occurrence of a given disease (e.g. an autoimmune, inflammatory autoimmune, cancer, infectious, immune, or other disease) or disease symptoms in a patient. As indicated above, the prevention may be complete (no detectable symptoms) or partial, such that fewer symptoms are observed than would likely occur absent treatment.

By “therapeutically effective dose or amount” as used herein is meant a dose that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

The term “diagnosis” refers to a relative probability that a disease (e.g. an autoimmune, inflammatory autoimmune, cancer, infectious, immune, or other disease) is present in the subject. Similarly, the term “prognosis” refers to a relative probability that a certain future outcome may occur in the subject with respect to a disease state. For example, in the context of the present invention, prognosis can refer to the likelihood that an individual will develop a disease (e.g. an autoimmune, inflammatory autoimmune, cancer, infectious, immune, or other disease), or the likely severity of the disease (e.g., duration of disease). The terms are not intended to be absolute, as will be appreciated by any one of skill in the field of medical diagnostics.

“Nucleic acid” or “oligonucleotide” or “polynucleotide” or grammatical equivalents used herein means at least two nucleotides covalently linked together. The term “nucleic acid” includes single-, double-, or multiple-stranded DNA, RNA and analogs (derivatives) thereof. Oligonucleotides are typically from about 5, 6, 7, 8, 9, 10, 12, 15, 25, 30, 40, 50 or more nucleotides in length, up to about 100 nucleotides in length. Nucleic acids and polynucleotides are a polymers of any length, including longer lengths, e.g., 200, 300, 500, 1000, 2000, 3000, 5000, 7000, 10,000, etc. In certain embodiments, the nucleic acids herein contain phosphodiester bonds. In other embodiments, nucleic acid analogs are included that may have alternate backbones, comprising, e.g., phosphoramidate, phosphorothioate, phosphorodithioate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and nonribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

A particular nucleic acid sequence also encompasses “splice variants.” Similarly, a particular protein encoded by a nucleic acid encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher, et al., J. Biol. Chem. 273(52):35095-35101 (1998).

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “probe” or “primer”, as used herein, is defined to be one or more nucleic acid fragments whose specific hybridization to a sample can be detected. A probe or primer can be of any length depending on the particular technique it will be used for. For example, PCR primers are generally between 10 and 40 nucleotides in length, while nucleic acid probes for, e.g., a Southern blot, can be more than a hundred nucleotides in length. The probe may be unlabeled or labeled as described below so that its binding to the target or sample can be detected. The probe can be produced from a source of nucleic acids from one or more particular (preselected) portions of a chromosome, e.g., one or more clones, an isolated whole chromosome or chromosome fragment, or a collection of polymerase chain reaction (PCR) amplification products. The length and complexity of the nucleic acid fixed onto the target element is not critical to the invention. One of skill can adjust these factors to provide optimum hybridization and signal production for a given hybridization procedure, and to provide the required resolution among different genes or genomic locations.

The probe may also be isolated nucleic acids immobilized on a solid surface (e.g., nitrocellulose, glass, quartz, fused silica slides), as in an array. In some embodiments, the probe may be a member of an array of nucleic acids as described, for instance, in WO 96/17958. Techniques capable of producing high density arrays can also be used for this purpose (see, e.g., Fodor (1991) Science 767-773; Johnston (1998) Curr. Biol. 8: R171-R174; Schummer (1997) Biotechniques 23: 1087-1092; Kern (1997) Biotechniques 23: 120-124; U.S. Pat. No. 5,143,854).

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe. Alternatively, a method using high affinity interactions may achieve the same results where one of a pair of binding partners binds to the other, e.g., biotin, streptavidin.

The terms “identical” or percent sequence “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site at ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. Employed algorithms can account for gaps and the like.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are wellknown in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively.

The phrase “selectively (or specifically) hybridizes to” refers to the binding, duplexing, or hybridizing of a molecule only to a particular nucleotide sequence with a higher affinity, e.g., under more stringent conditions, than to other nucleotide sequences (e.g., total cellular or library DNA or RNA).

The phrase “stringent hybridization conditions” refers to conditions under which a nucleic acid will hybridize to its target sequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent hybridization conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent hybridization conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., John Wiley & Sons.

Nucleic acids may be substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions.

An “inhibitory nucleic acid” is a nucleic acid (e.g. DNA, RNA, polymer of nucleotide analogs) that is capable of binding to a target nucleic acid (e.g. an mRNA translatable into PTPRS) and reducing transcription of the target nucleic acid (e.g. mRNA from DNA) or reducing the translation of the target nucleic acid (e.g. mRNA) or altering transcript splicing (e.g. single stranded morpholino oligo). A “morpholino oligo” may be alternatively referred to as a “morphlino nucleic acid” and refers to morpholine-containing nucleic acid nucleic acids commonly known in the art (e.g. phosphoramidate morpholinio oligo or a “PMO”). See Marcos, P., Biochemical and Biophysical Research Communications 358 (2007) 521-527. In some embodiments, the “inhibitory nucleic acid” is a nucleic acid that is capable of binding (e.g. hybridizing) to a target nucleic acid (e.g. an mRNA translatable into an RPTPS) and reducing translation of the target nucleic acid. The target nucleic acid is or includes one or more target nucleic acid sequences to which the inhibitory nucleic acid binds (e.g. hybridizes). Thus, an inhibitory nucleic acid typically is or includes a sequence (also referred to herein as an “antisense nucleic acid sequence”) that is capable of hybridizing to at least a portion of a target nucleic acid at a target nucleic acid sequence, An example of an inhibitory nucleic acid is an antisense nucleic acid. Another example of an inhibitory nucleic acid is siRNA or RNAi (including their derivatives or pre-cursors, such as nucleotide analogs). Further examples include shRNA, miRNA, shmiRNA, or certain of their derivatives or pre-cursors. In some embodiments, the inhibitory nucleic acid is single stranded. In other embodiments, the inhibitory nucleic acid is double stranded.

An “antisense nucleic acid” is a nucleic acid (e.g. DNA, RNA or analogs thereof) that is at least partially complementary to at least a portion of a specific target nucleic acid (e.g. a target nucleic acid sequence), such as an mRNA molecule (e.g. a target mRNA molecule) (see, e.g., Weintraub, Scientific American, 262:40 (1990)), for example antisense , siRNA, shRNA, shmiRNA, miRNA (microRNA). Thus, antisense nucleic acids are capable of hybridizing to (e.g. selectively hybridizing to) a target nucleic acid (e.g. target mRNA). In some embodiments, the antisense nucleic acid hybridizes to the target nucleic acid sequence (e.g. mRNA) under stringent hybridization conditions. In some embodiments, the antisense nucleic acid hybridizes to the target nucleic acid (e.g. mRNA) under moderately stringent hybridization conditions. Antisense nucleic acids may comprise naturally occurring nucleotides or modified nucleotides such as, e.g., phosphorothioate, methylphosphonate, and -anomeric sugar-phosphate, backbonemodified nucleotides. An “anti-PTPRS antisense nucleic acid” is an antisense nucleic acid that is at least partially complementary to at least a portion of a target nucleic acid sequence, such as an mRNA molecule, that codes at least a portion of the PTPRS. In some embodiments, an antisense nucleic acid is a morpholino oligo. In some embodiments, a morpholino oligo is a single stranded antisense nucleic acid, as is know in the art. In some embodiments, a morpholino oligo decreases protein expression of a target, reduces translation of the target mRNA, reduces translation initiation of the target mRNA, or modifies transcript splicing. In some embodiments, the morpholino oligo is conjugated to a cell permeable moiety (e.g. peptide). Antisense nucleic acids may be single or double stranded nucleic acids.

In the cell, the antisense nucleic acids may hybridize to the target mRNA, forming a double-stranded molecule. The antisense nucleic acids, interfere with the translation of the mRNA, since the cell will not translate a mRNA that is double-stranded. The use of antisense methods to inhibit the in vitro translation of genes is well known in the art (Marcus-Sakura, Anal. Biochem., 172:289, (1988)). Antisense molecules which bind directly to the DNA may be used.

Inhibitory nucleic acids can be delivered to the subject using any appropriate means known in the art, including by injection, inhalation, or oral ingestion. Another suitable delivery system is a colloidal dispersion system such as, for example, macromolecule complexes, nanocapsules, microspheres, beads, and lipid-based systems including oil-in-water emulsions, micelles, mixed micelles, and liposomes. An example of a colloidal system of this invention is a liposome. Liposomes are artificial membrane vesicles which are useful as delivery vehicles in vitro and in vivo. Nucleic acids, including RNA and DNA within liposomes and be delivered to cells in a biologically active form (Fraley, et al., Trends Biochem. Sci., 6:77, 1981). Liposomes can be targeted to specific cell types or tissues using any means known in the art. Inhibitory nucleic acids (e.g. antisense nucleic acids, morpholino oligos) may be delivered to a cell using cell permeable delivery systems (e.g. cell permeable peptides). In some embodiments, inhibitory nucleic acids are delivered to specific cells or tissues using viral vectors or viruses.

An “siRNA” refers to a nucleic acid that forms a double stranded RNA, which double stranded RNA has the ability to reduce or inhibit expression of a gene or target gene when the siRNA is present (e.g. expressed) in the same cell as the gene or target gene. The siRNA is typically about 5 to about 100 nucleotides in length, more typically about 10 to about 50 nucleotides in length, more typically about 15 to about 30 nucleotides in length, most typically about 20-30 base nucleotides, or about 20-25 or about 24-29 nucleotides in length, e.g., 20, 21, 22, 23, 24, 25, 26, 27, 28, 29, or 30 nucleotides in length. siRNA molecules and methods of generating them are described in, e.g., Bass, 2001, Nature, 411, 428-429; Elbashir et al., 2001, Nature, 411, 494-498; WO 00/44895; WO 01/36646; WO 99/32619; WO 00/01846; WO 01/29058; WO 99/07409; and WO 00/44914. A DNA molecule that transcribes dsRNA or siRNA (for instance, as a hairpin duplex) also provides RNAi. DNA molecules for transcribing dsRNA are disclosed in U.S. Pat. No. 6,573,099, and in U.S. Patent Application Publication Nos. 2002/0160393 and 2003/0027783, and Tuschl and Borkhardt, Molecular Interventions, 2:158 (2002).

The siRNA can be administered directly or siRNA expression vectors can be used to induce RNAi that have different design criteria. A vector can have inserted two inverted repeats separated by a short spacer sequence and ending with a string of T's which serve to terminate transcription.

Construction of suitable vectors containing the nucleic acid sequences employs standard ligation and restriction techniques, which are well understood in the art (see Maniatis et al., in Molecular Cloning: A Laboratory Manual, Cold Spring Harbor Laboratory, New York (1982)). Isolated plasmids, DNA sequences, or synthesized oligonucleotides are cleaved, tailored, and re-ligated in the form desired.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (i.e., prostate, lymph node, liver, bone marrow, blood cell, joint tissue, synovial tissue, synoviocytes, fibroblast-like synoviocytes, macrophage-like synoviocytes, immune cells, hematopoietic cells, fibroblasts, macrophages, T cells, etc.), among other factors. Representative biopsy techniques include excisional biopsy, incisional biopsy, needle biopsy, surgical biopsy, and bone marrow biopsy. Biopsy techniques are discussed, for example, in Harrison's Principles of Internal Medicine, Kasper, et al., eds., 16th ed., 2005, Chapter 70, and throughout Part V.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any method known in the art for conjugating an antibody to the label may be employed, e.g., using methods described in Hermanson, Bioconjugate Techniques 1996, Academic Press, Inc., San Diego.

The term “recombinant” when used with reference, e.g., to a cell, or nucleic acid, protein, or vector, indicates that the cell, nucleic acid, protein or vector has been modified by or is the result of laboratory methods. Thus, for example, recombinant proteins include proteins produced by laboratory methods. Recombinant proteins can include amino acid residues not found within the native (non-recombinant) form of the protein or can be include amino acid residues that have been modified, e.g., labeled.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. In some embodiments, antibodies or fragments of antibodies may be derived from different organisms, including humans, mice, rats, hamsters, camels, etc. Antibodies of the invention may include antibodies that have been modified or mutated at one or more amino acid positions to improve or modulate a desired function of the antibody (e.g. glycosylation, expression, antigen recognition, effector functions, antigen binding, specificity, etc.).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980 , WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun, 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

Techniques for conjugating therapeutic agents to antibodies are well known (see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)).

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

“PTPR” or “RPTP” or “rPTP” (all terms are equal) refer to receptor protein tyrosine phosphatases, which are found in nature as protein tyrosine phosphatases.

“PTPRS” refers to protein tyrosine phosphatase receptor type S (or sigma), which is a member of the protein tyrosine phosphatase (PTP) family. The amino acid sequence of PTPRS can be found, for example, at UniProtKB/Swiss-Prot Accession No. Q13332 and B0V2N1, and also SEQ ID NO:4. The nucleic acid sequence of PTPRS can be found, for example, at GenBank Accession No. NC_000019.9 and. PTPRS includes an intracellular domain, e.g., amino acid residues 1304-1948 of SEQ ID NO:8 or amino acid residues 1279-1907 of SEQ ID NO:4, a transmembrane domain, e.g., amino acid residues 1283-1303 of SEQ ID NO:8 or amino acid residues 1258-1278 of SEQ ID NO:4, and an extracellular domain, e.g., SEQ ID NO:9 or SEQ ID NO:10. The term transmembrane domain refers to the portion of a protein or polypeptide that is embedded in and, optionally, spans a membrane. The term intracellular domain refers to the portion of a protein or polypeptide that extends into the cytoplasm of a cell. The term extracellular domain refers to the portion of a protein or polypeptide that extends into the extracellular environment. The extracellular domain of PTPRS includes immunoglobulin-like domain 1 (Ig1), immunoglobulin-like domain 2 (Ig2) and immunoglobulin-like domain 2 (Ig3). The amino acid sequence of Ig1 includes amino acid residues 30 to 127 of SEQ ID NO:4 or amino acid residues 30-127 of SEQ ID NO:8, or the amino acid sequence EEPRFIKEPKDQIGVSGGVASFVCQATGDPKPRVTWNKKGKKVNSQRFETIEFDESAGA VLRIQPLRTPRDENVYECVAQNSVGEITVHAKLTVLRE (SEQ ID NO:1) or the amino acid sequence of SEQ ID NO:5. The amino acid sequence of Ig2 includes amino acid residues 128 to 231 of SEQ ID NO:4, or amino acid residues 128-244 of SEQ ID NO:8, or the amino acid sequence DQLPSGFPNIDMGPQLKVVERTRTATMLCAASGNPDPEITWFKDFLPVDPSASNGRIKQL RSETFESTPIRGALQIESSEETDQGKYECVATNSAGVRYSSPANLYVRVRRVA (SEQ ID NO:2) or the amino acid sequence of SEQ ID NO:6. The amino acid sequence of Ig3 includes amino acid residues 232 to 321 of SEQ ID NO:4, or amino acid residues 245-334 of SEQ ID NO:8 or the amino acid sequence PRFSILPMSHEIMPGGNVNITCVAVGSPMPYVKWMQGAEDLTPEDDMPVGRNVLELTD VKDSANYTCVAMSSLGVIEAVAQITVKSLPKA (SEQ ID NO:3) or the amino acid sequence of SEQ ID NO:7.

A “protein level of an RPTP” refers to an amount (relative or absolute) of RPTP in its protein form (as distinguished from its precursor RNA form). A protein of an RPTP may include a full-length protein (e.g. the protein translated from the complete coding region of the gene, which may also include post-translational modifications), functional fragments of the full length protein (e.g. sub-domains of the full length protein that possess an activity or function in an assay), or protein fragments of the RPTP, which may be any peptide or oligopeptide of the full length protein.

An “RNA level of an RPTP” refers to an amount (relative or absolute) of RNA present that may be translated to form an RPTP. The RNA of an RPTP may be a full-length RNA sufficient to form a full-length RPTP. The RNA of an RPTP may also be a fragment of the full length RNA thereby forming a fragment of the full length RPTP. The fragment of the full length RNA may form a functional fragment of the RPTP. In some embodiments, the RNA of an RPTP includes all splice variants of an RPTP gene.

An “autoimmune therapeutic agent” is a molecule (e.g. antibody, nucleic acid, inhibitory nucleic acid, synthetic chemical, small chemical molecule) that treats or prevents an autoimmune disease when administered to a subject in a therapeutically effective dose or amount. In some embodiments, an autoimmune therapeutic agent is an RPTP binding agent.

An “RPTP binding agent” is a molecule that binds (e.g. preferentially binds) to RPTP, RNA that is translatable to RPTP, or DNA that is transcribable to an RNA that is translatable to an RPTP. Where the molecule preferentially binds, the binding is preferential as compared to other macromolecular biomolecules present in an organism or cell. A compound preferentially binds to as compared to other macromolecular biomolecules present in an organism or cell, for example, when the preferential binding is 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10000-fold, 100,000-fold, 1,000,000-fold greater.

An agent may “target” an RPTP, a nucleic acid (e.g. RNA or DNA) encoding an RPTP, or a protein of an RPTP, by binding (e.g. preferentially binding) to the RPTP, nucleic acid (e.g. RNA or DNA) encoding an RPTP, or protein of an RPTP. Optionally, the RPTP is PTPRS. An agent preferentially binds to a molecule, for example, when the binding to the targeted molecule is greater than the binding to other molecules of a similar form. In some embodiments, the preferential binding is 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10000 fold, 100,000-fold, 1,000,000-fold greater. In some embodiments, an agent targets an RPTP, a nucleic acid (e.g. RNA or DNA) of an RPTP, or a protein of an RPTP when a binding assay or experiment (e.g. gel electrophoresis, chromatography, immunoassay, radioactive or non-radioactive labeling, immunoprecipitation, activity assay, etc.) reveals only an interaction or primarily an interaction with a single RPTPS, a nucleic acid (e.g. RNA or DNA) of a single RPTP, or a protein of a single RPTP. An agent may also “target” an RPTP, a nucleic acid (e.g. RNA or DNA) of an RPTP, or a protein of an RPTPS by binding to the RPTP, nucleic acid (e.g. RNA or DNA) of an RPTP, or protein of an RPTP, by decreasing or increasing the amount of RPTP in a cell or organism relative to the absence of the agent, or decreasing the interaction between the RPTP with a physiological or natural ligand. A person having ordinary skill in the art, using the guidance provided herein, may easily determine whether an agent decreases or increases the amount of an RPTP in a cell or organism.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

Provided herein are PTPRS de-clustering agents. As used herein, the terms “PTPRS de-clustering agent” and the like refer to an agent (e.g., small molecules, proteins including antibodies, and the like) capable of causing a reduction in the level of dimerization, oligomerization or clustering of PTPRS proteins. Without wishing to be bound by any theory, it is believed that clustering of PTPRS can give rise to an inactive dimeric form. Accordingly, the action of a PTPRS de-clustering agent results in monomeric PTPRS which regains activity relative to the clustered (e.g., dimerized or oligomerized) form of PTPRS. Optionally, the PTPRS de-clustering agent is a non-enzymatic recombinant protein comprising an amino acid sequence of an extracellular domain of PTPRS or a subsequence, portion, homologue, variant or derivative thereof, as described herein. Optionally, the non-enzymatic recombinant protein is the extracellular domain of PTPRS. Without being limited to any particular theory, the extracellular domain of PTPRS or portions thereof displaces PTPRS from HS. This can activate PTPRS and lead to dephosphorylation of beta-catenin and other substrates and inhibition of downstream FLS invasiveness and pro-inflammatory actions. This is supported by the examples and data provided herein. Optionally, the PTPRS de-clustering agent is an anti-PRPRS antibody or fragment thereof. Optionally, the PTPRS de-clustering agent is an anti-PTPRS aptamer. Optionally, the PTPRS de-clustering agent binds heparan sulfate. Optionally, the PTPRS de-clustering agent is an anti-heparan sulfate antibody or fragment thereof. Optionally, the PTPRS de-clustering agent is an anti-heparan sulfate aptamer. Optionally, the PTPRS de-clustering agent is not chondroitin sulfate. The PTPRS is, optionally, not a chondroitin sulfate mimetic or an agent that has the same or similar mechanism of action as chondroitin sulfate. In other embodiments, the PTPRS de-clustering agent is a chondroitin sulfate mimetic.

Provided herein are non-enzymatic recombinant proteins comprising an amino acid sequence of an extracellular domain of PTPRS, or a subsequence, portion, homologue, variant or derivative thereof. As used herein, the term “non-enzymatic recombinant protein” refers to a recombinant protein that does not have enzymatic activity (e.g., the protein does not function as a biological catalyst). Thus, in some embodiments, the non-enzymatic recombinant proteins comprising an amino acid sequence of an extracellular domain of PTPRS provided herein include only extracellular domain portions of the PTPRS and not the enzymatic portions of the PTPRS. In embodiments, the non-enzymatic recombinant proteins comprising an amino acid sequence of an extracellular domain of PTPRS provided herein include only extracellular domain portions of the PTPRS and not the enzymatic portions of the PTPRS or the transmembrane portions of the PTPRS. In some embodiments, the non-enzymatic recombinant proteins comprising an amino acid sequence of an extracellular domain of PTPRS include two or more extracellular domain of PTPRS linked together (e.g. linked by an amino acid linker such as an amino acid linker of at least 2, at least 3, at least 5, at least 10, about 2 to 50 or 100 amino acids, about 3 to 50 or 100 amino acids, or about 2, 3,4 5, 6, 7, 8, 9, 10, 20, 30, 40, 50, 60, 70, 80, 90, or 100 amino acides wherein the amino acid sequence is designed to not interfere with extracellular domain of PTPRS ligand binding). The term “extracellular domain of PTPRS” can include subsequences, portions, homologues, variants or derivatives of the extracellular domain of PTPRS. Thus, the non-enzymatic recombinant protein can comprise a portion of the extracellular domain of PTPRS, e.g., the protein comprises one or more immunoglobulin-like domains of PTPRS, e.g., SEQ ID NO:1, SEQ ID NO:2, SEQ ID NO:3, SEQ ID NO:5, SEQ ID NO:6 and/or SEQ ID NO:7, or subsequences, portions, homologues, variants or derivatives thereof. The extracellular domain of PTPRS is typically capable of binding (e.g specifically binding) to a PTPRS ligand such as heparan sulfate. Optionally, the extracellular domain of PTPRS comprises one or more of PTPRS immunoglobulin-like domain 1 (Ig1), immunoglobulin-like domain 2 (Ig2) and immunoglobulin-like domain 2 (Ig3), or a subsequence, portion, homologue, variant or derivative thereof. Optionally, the extracellular domain of PTPRS comprises one or both of PTPRS immunoglobulin-like domain 1 (Ig1) and immunoglobulin-like domain 2 (Ig2) or a subsequence, portion, homologue, variant or derivative thereof.

Optionally, the protein comprises Ig1 amino acid residues 39 to 124 of SEQ ID NO:4, or a subsequence, portion, homologue, variant or derivative thereof. Optionally, the protein comprises an amino acid sequence set forth as: EEPRFIKEPKDQIGVSGGVASFVCQATGDPKPRVTWNKKGKKVNSQRFETIEFDESAGA VLRIQPLRTPRDENVYECVAQNSVGEITVHAKLTVLRE(SEQ ID NO:1) or set forth as SEQ ID NO:5, or a subsequence, portion, homologue, variant or derivative thereof.

Optionally, the protein comprises Ig2 amino acid residues 152 to 233 of SEQ ID NO:4, or a subsequence, portion, homologue, variant or derivative thereof. Optionally, the protein comprises an amino acid sequence set forth as:

DQLPSGFPNIDMGPQLKVVERTRTATMLCAASGNPDPEITWFKDFLPVDPSASNGRIKQL RSETFESTPIRGALQIESSEETDQGKYECVATNSAGVRYSSPANLYVRVRRVA (SEQ ID NO:2) or set forth as SEQ ID NO:6, or a subsequence, portion, homologue, variant or derivative thereof.

Optionally, the protein comprises Ig3 amino acid residues 259-327 of SEQ ID NO:4, or a subsequence, portion, homologue, variant or derivative thereof. Optionally, the protein comprises an amino acid sequence set forth as: PRFSILPMSHEIMPGGNVNITCVAVGSPMPYVKWMQGAEDLTPEDDMPVGRNVLELTD VKDSANYTCVAMSSLGVIEAVAQITVKSLPKA (SEQ ID NO:3), or set forth as SEQ ID NO:7, or a subsequence, portion, homologue, variant or derivative thereof.

In some embodiments, the non-enzymatic recombinant protein comprising an amino acid sequence of an extracellular domain of PTPRS or portion thereof lacks a transmembrane domain and/or lacks an intracellular domain. In some embodiments, the non-enzymatic recombinant protein comprising an amino acid sequence of an extracellular domain of PTPRS or portion thereof lacks a transmembrane domain. In some embodiments, the non-enzymatic recombinant protein comprising an amino acid sequence of an extracellular domain of PTPRS or portion thereof lacks an intracellular domain.

Optionally, the provided PTPRS de-clustering agent binds (e.g. specifically binds) heparan sulfate. Optionally, the PTPRS de-clustering agent prevents oligomerization or clustering of PTPRS proteins; for example, the PTPRS de-clustering agent prevents dimerization of PTPRS proteins. Optionally, the PTPRS de-clustering agent modulates PTPRS activity; for example, the PTPRS de-clustering agent increases the phosphatase activity of PTPRS.

Provided herein are compositions including the agents provided herein. Provided herein are pharmaceutical compositions including a PTPRS de-clustering agent and a pharmaceutically acceptable excipient. Provided compositions can include a single agent or more than one agent. The provided compositions are, optionally, suitable for formulation and administration in vitro or in vivo. Optionally, the compositions comprise one or more of the provided agents and a pharmaceutically acceptable carrier. Suitable carriers and their formulations are described in Remington: The Science and Practice of Pharmacy, 21st Edition, David B. Troy, ed., Lippicott Williams & Wilkins (2005). By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. If administered to a subject, the carrier is optionally selected to minimize degradation of the active ingredient and to minimize adverse side effects in the subject.

The term “pharmaceutically acceptable salts” or “pharmaceutically acceptable carrier” is meant to include salts of the active compounds which are prepared with relatively nontoxic acids or bases, depending on the particular substituents found on the compounds described herein. When compounds of the present application contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present application contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Other pharmaceutically acceptable carriers known to those of skill in the art are suitable for compositions of the present application.

The compositions for administration will commonly comprise an agent as described herein dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the subject's needs.

Solutions of the active compounds as free base or pharmacologically acceptable salt can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms.

Pharmaceutical compositions can be delivered via intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations and appropriate drug stabilizers, if required, may be included in the formulation. Various commercial nasal preparations are known and can include, for example, antibiotics and antihistamines.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In some embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such compositions is such that a suitable dosage can be obtained

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion

Sterile injectable solutions can be prepared by incorporating the active compounds or constructs in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more, or highly, concentrated solutions for direct injection is also contemplated. DMSO can be used as solvent for extremely rapid penetration, delivering high concentrations of the active agents to a small area.

The formulations of compounds can be presented in unit-dose or multi-dose sealed containers, such as ampules and vials. Thus, the composition can be in unit dosage form. In such form the preparation is subdivided into unit doses containing appropriate quantities of the active component. Thus, the compositions can be administered in a variety of unit dosage forms depending upon the method of administration. For example, unit dosage forms suitable for oral administration include, but are not limited to, powder, tablets, pills, capsules and lozenges.

Compositions can be formulated to provide quick, sustained or delayed release after administration by employing procedures known in the art. Certain carriers may be more preferable depending upon, for instance, the route of administration and concentration of composition being administered. Suitable formulations for use in the provided compositions can be found in Remington: The Science and Practice of Pharmacy, 21st Edition, David B. Troy, ed., Lippicott Williams & Wilkins (2005).

Provided herein are kits comprising one or more of the provided compositions and instructions for use. Optionally, the kit comprises one or more doses of an effective amount of a composition comprising a PTPRS de-clustering agent. Optionally, the kit comprises a non-enzymatic recombinant protein comprising an amino acid sequence of an extracellular domain of PTPRS or a subsequence, portion, homologue, variant or derivative thereof. Optionally, the kit comprises one or more portions of the extracellular domain of PTPRS. Optionally, the composition or protein is present in a container (e.g., vial or packet). Optionally, the kit comprises one or more additional agents for treating or preventing one or more symptom of an inflammatory and/or autoimmune disease. Optionally, the kit comprises a means of administering the composition, such as, for example, a syringe, needle, tubing, catheter, patch, and the like. The kit may also comprise formulations and/or materials requiring sterilization and/or dilution prior to use.

The compositions and agents as described herein are useful for both prophylactic and therapeutic treatment. For prophylactic use, a therapeutically effective amount of the agents described herein are administered to a subject prior to or during early onset (e.g., upon initial signs and symptoms of an autoimmune disease). Therapeutic treatment involves administering to a subject a therapeutically effective amount of the agents described herein after diagnosis or development of disease.

The provided proteins, agents and compositions are for use in the treatment of a subject who has or is at risk of developing an autoimmune disease, including for example arthritis such as rheumatoid arthritis or osteoarthritis. Optionally, the proteins, agents, and compositions are for use in the treatment of a subject who has or is at risk of developing an extracellular matrix disease and/or a fibroblast-mediated disease. As used herein, the term “extracellular matrix disease” refers to a condition, disorder or disease, associated with the extracellular matrix (ECM) or one or more components of the extracellular matrix. The extracellular matrix provides structural support to cells in addition to being involved in other biological functions including, but not limited to, intracellular communication. Components of the extracellular matrix include, but are not limited to, proteoglycans (e.g., heparan sulfate, chondroitin sulfate, and keratin sulfate), non-proteoglycan polysaccharaides (e.g., hyaluronic acid), fibers, collagen, elastin, fibronectin and laminin. The extracellular matrix also serves as a depot for signaling molecules such as growth factors and cytokines. Extracellular matrix diseases include diseases associated with the dysregulation of one or more functions of the ECM (e.g., dysregulated intracellular communication and/or movement) or dysreguation of one or more components of the ECM (e.g., increased or decreased activity and/or production of one or more components of the ECM). Extracellular matrix diseases also include diseases associated with altered degradation and remodeling of the ECM and diseases associated with altered (e.g., increased or decreased) accumulation of agents, e.g., immunocomplexes and other immune products, in the ECM. Extracellular matrix diseases include, but are not limited to, atherosclerosis, cancer, amyloid diseases, glomerular diseases, mesangial diseases, inflammatory conditions, and developmental disorders. As used herein, the term “fibroblast-mediated disease” refers to a condition, disorder, or disease, associated with fibroblast cell activity or movement. Fibroblasts are a type of cell involved in the synthesis of the ECM and collagen and are the major cell type of connective tissue. Types of fibroblasts include, but are not limited to, synovial fibroblasts, dermal fibroblasts, and interstitial fibroblasts. The main function of fibroblasts is to maintain the integrity of connective tissue by continuously secreting components of the ECM. Fibroblast-mediated diseases include diseases associated with the altered activity and/or movement of fibroblasts. Thus, for example, a fibroblast-mediated disease includes diseases associated with altered fibroblast migration or altered fibroblast activity. Fibroblast activities include, but are not limited to, collagen production, glycosaminoglycan production, reticular and elastic fiber production, cytokine production, and glycoprotein production. Thus, fibroblast-mediated diseases include diseases associated with altered production by fibroblasts of one or more of collagen, glycosaminoglycans, reticular and elastic fibers, cytokines, and glycoproteins.

Provided herein are methods of modulating PTPRS activity in a subject, the method comprising administering to the subject an effective amount of a PTPRS de-clustering agent, wherein administration modulates PTPRS activity in the subject. Also provided are methods of treating, preventing, and/or ameliorating an autoimmune disease or disorder in a subject in need thereof. Specifically, provided is a method of treating an autoimmune disease in a subject, the method comprising administering to the subject a therapeutically effective amount of a PTPRS de-clustering agent, wherein administration treats the autoimmune disease in the subject. Also provided is a method of treating an autoimmune disease in a subject, the method comprising administering to the subject a therapeutically effective amount of a compound that increases PTPRS phosphatase activity, wherein administration treats the autoimmune disease in the subject. Autoimmune diseases or disorders include, but are not limited to, inflammatory autoimmune diseases. Optionally, the autoimmune disease is arthritis, rheumatoid arthritis, psoriatic arthritis, juvenile idiopathic arthritis, scleroderma, systemic scleroderma, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome,vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, or allergic asthma. Optionally, the autoimmune disease is arthritis, Crohn's disease, scleroderma, or rheumatoid arthritis. Optionally, the compound is a PTPRS de-clustering agent. Optionally, the PTPRS de-clustering agent is not chondroitin sulfate. Optionally, the de-clustering agent is not chondroitin sulfate, a chondroitin sulfate mimetic or an agent that has the same or similar mechanism of action as chondroitin sulfate. Optionally, the PTPRS de-clustering agent is an anti-PTPRS antibody or fragment thereof, an anti-heparan sulfate antibody, or a chondroitin sulfate mimetic. Optionally, as described throughout, the PTPRS de-clustering agent can be a non-enzymatic recombinant protein comprising an amino acid sequence of an extracellular domain of PTPRS or a subsequence, portion, homologue variant or derivative thereof.

The methods include administering an effective amount of the provided agents and compositions, wherein administering the effective amount of the composition treats or prevents the autoimmune disease in the subject. Administration of a composition disclosed herein can be a systemic or localized administration. For example, treating a subject having an inflammatory autoimmune disorder can include administering an oral or injectable form of the pharmaceutical composition on a daily basis or otherwise regular schedule. Optionally, the agents and composition are formulated for administration can be formulated for delivery to synovial fluid and/or for delivery to fibroblast-like synoviocytes. In some embodiments, the treatment is only on an as-needed basis, e.g., upon appearance of inflammatory autoimmune disease symptoms.

Also provided are methods of decreasing fibroblast activity in a subject. The methods include administering to the subject a therapeutically effective amount of a PTPRS de-clustering agent, wherein administration decreases fibroblast activity in the subject. Optionally, the de-clustering agent is not chondroitin sulfate, a chondroitin sulfate mimetic or an agent that has the same or similar mechanism of action as chondroitin sulfate. In some embodiments, the de-clustering agent is a chondroitin sulfate mimetic. Optionally, the PTPRS de-clustering agent is a non-enzymatic recombinant protein as provided herein. Optionally, the PTPRS de-clustering agent binds heparan sulfate. Optionally, the PTPRS de-clustering agent is an anti-PTPRS antibody or fragment thereof or an anti-heparan sulfate antibody or fragment thereof. Optionally, the fibroblast activity comprises fibroblast migration. Optionally, the fibroblast activity comprises collagen production, glycosaminoglycan production, reticular and elastic fiber production, cytokine production, chemokine production, glycoprotein production or combinations thereof. Optionally, the fibroblast activity comprises extracellular matrix production. Fibroblasts include, but are not limited to, synovial fibroblasts, dermal fibroblasts, and interstitial fibroblasts. Optionally, the fibroblasts are synovial fibroblasts.

Optionally, the subject has a fibroblast-mediated disease. Thus, provided are methods of treating a fibroblast mediated disease in a subject. The methods include administering to the subject a therapeutically effective amount of a PTPRS de-clustering agent, wherein administration treats the fibroblast-mediated disease in the subject. Optionally, the de-clustering agent is not chondroitin sulfate, a chondroitin sulfate mimetic or an agent that has the same or similar mechanism of action as chondroitin sulfate. In some embodiments, the de-clustering agent is a chondroitin mimetic. Optionally, the PTPRS de-clustering agent is a non-enzymatic recombinant protein as provided herein. Optionally, the PTPRS de-clustering agent binds heparan sulfate. Optionally, the PTPRS de-clustering agent is an anti-PTPRS antibody or fragment thereof or an anti-heparan sulfate antibody or fragment thereof. Fibroblast-mediated diseases include, but are not limited to, fibrosis and fibroblast-mediated autoimmune diseases. The fibrosis can be, for example, pulmonary fibrosis, idiopathic pulmonary fibrosis, liver fibrosis, endomyocardial fibrosis, atrial fibrosis, mediastinal fibrosis, myelofibrosis, retroperitoneal fibrosis, nephrogenic systemic fibrosis, skin fibrosis, or arthrofibrosis. The fibroblast-mediated autoimmune disease can be, for example, Crohn's disease, arthritis, rheumatoid arthritis, and scleroderma.

Provided herein are methods of modulating extracellular matrix in a subject, the method comprising administering to the subject an effective amount of the non-enzymatic recombinant protein provided herein, wherein administration modulates the extracellular matrix in the subject. Optionally, the method does not include administration of chondroitin sulfate, a chondroitin sulfate mimetic or an agent that has the same or similar mechanism of action as chondroitin sulfate. Modulation of the extracellular matrix includes, for example, modulation of one or more components of the extracellular matrix. Optionally, the extracellular matrix component is selected from the group consisting of a proteoglycan, polysaccharide or fiber. Optionally, the extracellular matrix component is a proteoglycan, e.g., heparan sulfate or chondroitin sulfate. Optionally, the extracellular matrix component is heparan sulfate. Optionally, the subject has an extracellular matrix disease. Extracellular matrix diseases are known and include, but are not limited to, atherosclerosis, cancer, an amyloid disease, an inflammatory condition, and a developmental disorder. Optionally, the amyloid disease is Alzheimer's disease or inflammation-related AA amyloidosis. Optionally, the inflammatory condition is osteoarthritis, systemic scleroderma, or lupus.

The herein provided methods that include the treatment of subjects with an inflammatory condition, autoimmune disease, fibroblast-mediated disease, or extracellular matrix disease can include administration of one or more additional agents that treat or prevent the inflammatory condition or autoimmune disease. For example, the provided methods can further include administration of and effective amount of one or more of anti-inflammatory agents. Suitable additional agents for use in the provided methods include, but are not limited to, analgesics, non-steroidal anti-inflammatory drugs, disease-modifying anti-rheumatic drugs, corticosteroids, and vitamin D analogues. Exemplary disease-modifying anti-rheumatic drugs for treating or preventing rheumatoid arthritis include, but are not limited to, azathioprine, cyclosporine A, D-penicillamine, gold salts, hydroxychloroquine, leflunomide, methotrexate (MTX), minocycline, sulfasalazine (SSZ), and cyclophosphamide.

Combinations of agents or compositions can be administered either concomitantly (e.g., as a mixture), separately but simultaneously (e.g., via separate intravenous lines) or sequentially (e.g., one agent is administered first followed by administration of the second agent). Thus, the term combination is used to refer to concomitant, simultaneous or sequential administration of two or more agents or compositions. The course of treatment is best determined on an individual basis depending on the particular characteristics of the subject and the type of treatment selected. The treatment, such as those disclosed herein, can be administered to the subject on a daily, twice daily, bi-weekly, monthly or any applicable basis that is therapeutically effective. The treatment can be administered alone or in combination with any other treatment disclosed herein or known in the art. The additional treatment can be administered simultaneously with the first treatment, at a different time, or on an entirely different therapeutic schedule (e.g., the first treatment can be daily, while the additional treatment is weekly).

According to the methods provided herein, the subject is administered an effective amount of one or more of the agents provided herein. The terms effective amount and effective dosage are used interchangeably. The term effective amount is defined as any amount necessary to produce a desired physiologic response (e.g., reduction of inflammation). Effective amounts and schedules for administering the agent may be determined empirically by one skilled in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)).

Optionally, the provided methods of treatment or method of modulating PTPRS activity or function in a subject further includes obtaining a biological sample from the subject and determining whether the subject has an altered RNA level or an altered protein level of an PTPRS as compared to a control, an altered RNA level or altered protein level indicating the subject has or is at risk of developing an inflammatory condition, autoimmune disease, fibroblast-mediated disease or extracellular matrix disease. Optionally, the altered level is an elevated level as compared to a control. A control sample or value refers to a sample that serves as a reference, usually a known reference, for comparison to a test sample. For example, a test sample can be taken from a patient suspected of having autoimmune disease and compared to samples from a subject known to have an autoimmune disease or a known normal (non-disease) subject. A control can also represent an average value gathered from a population of similar individuals, e.g., autoimmune disease patients or healthy individuals with a similar medical background, same age, weight, etc. A control value can also be obtained from the same individual, e.g., from an earlier-obtained sample, prior to disease, or prior to treatment.

Thus, also provided are methods of determining whether a subject has or is at risk for developing an inflammatory condition, autoimmune disease, fibroblast-mediated disease or extracellular matrix disease comprising obtaining a biological sample from the subject and determining whether the subject has an elevated RNA level or an elevated protein level of an PTPRS, an elevated RNA level or elevated protein level indicating the subject has or is at risk of developing an autoimmune disease, inflammatory disease, fibroblast-mediated disease or extracellular matrix disease. Optionally, the provided methods further comprise selecting a subject with an autoimmune disease. Optionally, the autoimmune disease is an inflammatory autoimmune disease, e.g., arthritis or rheumatoid arthritis. As used herein, biological samples include, but are not limited to, cells, tissues and bodily fluids. Bodily fluids that used to evaluate the presence, absence or level of PTPRS RNA or protein include without limitation whole blood, plasma, urine, serum, tears, lymph, bile, cerebrospinal fluid, interstitial fluid, aqueous or vitreous humor, colostrum, sputum, amniotic fluid, saliva, a bronchioaviolar lavage sample, perspiration, transudate, exudate, and synovial fluid. Optionally, the biological sample is derived from a joint tissue or bodily fluid. Optionally, the provided methods further comprise isolating cells from the joint tissue or bodily fluid thereby forming an isolated cell sample. Such isolated cell samples can comprise synoviocytes, fibroblasts, hematopoetic cells, macrophages, leukocytes, T-cells or a combination thereof. Optionally, the synoviocytes are fibroblast-like synoviocytes or macrophage-like synoviocytes. Optionally, the isolated cell sample comprises fibroblast-like synoviocytes.

Methods for detecting and identifying nucleic acids and proteins and interactions between such molecules involve conventional molecular biology, microbiology, and recombinant DNA techniques within the skill of the art. Such techniques are explained fully in the literature (see, e.g., Sambrook, Fritsch & Maniatis, Molecular Cloning: A Laboratory Manual, Second Edition 1989, Cold Spring Harbor Laboratory Press, Cold Spring Harbor, N.Y.; Animal Cell Culture, R. I. Freshney, ed., 1986).

Methods for detecting RNA are largely cumulative with the nucleic acid detection assays and include, for example, Northern blots, RT-PCR, arrays including microarrays and sequencing including high-throughput sequencing methods. In some embodiments, a reverse transcriptase reaction is carried out and the targeted sequence is then amplified using standard PCR. Quantitative PCR (qPCR) or real time PCR (RT-PCR) is useful for determining relative expression levels, when compared to a control. Quantitative PCR techniques and platforms are known in the art, and commercially available (see, e.g., the qPCR Symposium website, available at qpersymposium.com). Nucleic acid arrays are also useful for detecting nucleic acid expression. Customizable arrays are available from, e.g., Affymatrix. Optionally, methods for detecting RNA include sequencing methods. RNA sequencing are known and can be performed with a variety of platforms including, but not limited to, platforms provided by Illumina, Inc., (La Jolla, Calif.) or Life Technologies (Carlsbad, Calif.). See, e.g., Wang, et al., Nat Rev Genet. 10(1):57-63 (2009); and Martin, Nat Rev Genet. 12(10):671-82 (2011).

Protein levels or concentration can be determined by methods standard in the art for quantitating proteins, such as Western blotting, ELISA, ELISPOT, immunoprecipitation, immunofluorescence (e.g., FACS), immunohistochemistry, immunocytochemistry, etc., as well as any other method now known or later developed for quantitating protein in or produced by a cell.

Also provided are methods of identifying a candidate PTPRS de-clustering agent, the method comprising contacting a test agent with clustered PTPRS peptides and detecting de-clustering of the PTPRS peptides, thereby identifying a candidate PTPRS de-clustering agent. Optionally, the method of identifying a candidate PTPRS de-clustering agent includes contacting a test agent with PTPRS and heparan sulfate and determining whether the test agent inhibits or reduces binding of the PTPRS to heparan sulfate, inhibition or reduction of binding indicating the test agent is a PTPRS de-clustering agent. Optionally, the test agent is a nucleic acid, peptide, antibody or small molecule. Optionally, the clustered PTPRS forms part of a cell membrane or cell. The methods of identifying PTPRS de-clustering agents may be performed in vitro, in situ or in vivo. A test agent can be identified as a PTPRS de-clustering agent using the methods described in the example below and others known to those of skill in the art. See, e.g., see Sambrook et al., Molecular Cloning: A Laboratory Manual, 3rd Ed., Cold Spring Harbor Press, Cold Spring Harbor, N.Y. (2001). Thus, de-clustering of clustered PTPRS can be performed using any appropriate method known in the art. Various methods are available to assess whether an agent is effective at declustering PTPRS. For example, a cell-based readout assay for PTPRS function can be used. Declustering of PTPRS can be observed by detecting a decrease in tyrosine phosphorylation of PTPRS substrates or downstream signaling intermediates. Declustering can also be observed by detecting a decrease in migration or invasion of fibroblasts (e.g., FLS). Another assays include, but are not limited to, FRET-based assays, and gel-filtration. For example, in such assays, cells can be treated with HS to induce PTPRS clustering, and then treated with the test agents to test for declustering of PTPRS. Similarly, inhibition binding of PTPRS to heparan sulfate can be detected using any appropriate method known in the art. For example, an agent can be identified as an agent that inhibits or reduces binding of PTPRS to heparan sulfate by performing an assay in which binding of PTPRS to heparan sulfate can be detected (e.g., an immunoassay). The agent inhibits or reduces binding of PTPRS to heparan sulfate if binding of PTPRS to heparan sulfate can be detected in the absence of the agent but is no longer detected or binding is reduced in the presence of the agent. Optionally, binding can be detected by determining whether the agent to be tested competitively inhibits heparan sulfate from binding to PTPRS. Optionally, an agent can be identified as an agent that inhibits or reduces binding of PTPRS to heparan sulfate by performing an assay that measures the function or activity of PTPRS.

By way of example, a test agent can be identified as a PTPRS de-clustering agent by determining if the agent reduces the severity of one or more symptoms of the autoimmune or inflammatory disease or condition. Thus, by way of example, in the provided screening methods, the contacting step comprises administering the agent to a subject with an autoimmune or inflammatory disease and the determining step comprises determining whether the agent prevents or reduces one or more symptoms of the disease in a subject. Optionally, the disease is arthritis or rheumatoid arthritis. Such screening methods can be carried out using, for example, animal models of inflammatory and autoimmune disease, which can be obtained from commercial laboratories (e.g., Jackson Laboratory, 600 Main Street, Bar Harbor, Me. 04609 USA). Such screening methods can also be carried out using the mouse models and methods described in the example below.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

A number of embodiments have been described. Nevertheless, it will be understood that various modifications may be made. Accordingly, other embodiments are within the scope of the claims.

### EXAMPLE

### Example 1. Functional Interactions Between PTPRS and Proteoglycans in Fibroblast-Like Synoviocytes

Protein Tyrosine Phosphatase, Receptor Type, S or Sigma (PTPRS) has an extracellular domain that includes multiple fibronectin type III-like domains and immunoglobulin-like (Ig) domains. The two most external Ig domains (called Ig1 and Ig2) interact with nanomolar affinity with HS—(Heparan sulfate) and CS—(chondroitin-sulfate) containing PG (proteoglycan). The interaction of PTPRS with HS-PG (proteoglycan) (which are layered on the cell surface of most cell types) induces a clustered topology of PTPRS, which inhibits the phosphatase action and promotes signaling. On the contrary, CS-PG (enriched in the ECM surrounding neurons and other cells) effectively competes with HS-PG inducing a declustered conformation, which is active and leads to signaling inhibition. In certain experiments, PTPRS knockout mice were used. These mice showed a phenotype of decreased body weight and brain size, deficits in learning memory, nerve functions, increased spinal cord regeneration after hemisection and contusion injuries and increased axon formation through inhibitory barriers (CSPG).

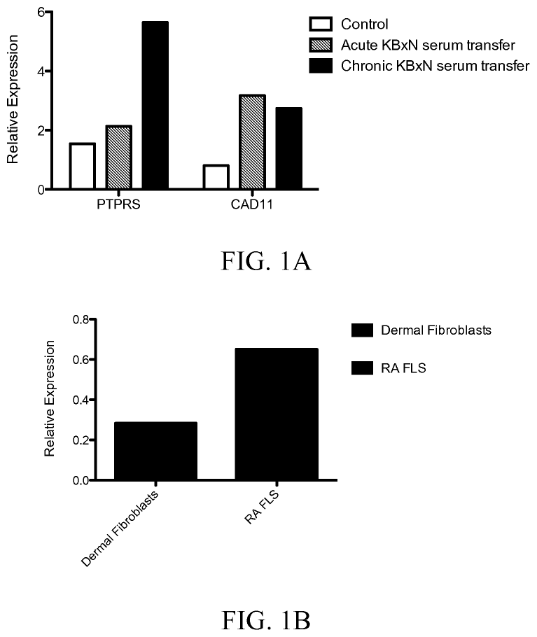

Mice that express the T cell receptor (TCR) transgene KRN and the MHC class II molecule A(g7) (K/BxN mice) develop severe inflammatory arthritis. The serum from these mice causes arthritis in other mouse strains. To determine whether PTPRS is involved in arthritis, expression of PTPRS in mice given K/BxN serum was evaluated. FIGS. 1A and B are graphs depicting expression of PTPRS in fibroblast-like synoviocytes (FLS). FIG. 1A shows expression in mice. Primary CD90+CD11b-CD3-CD45-mFLS were isolated from joint tissue 7 days after acute K/BxN serum transfer (single administration of 150 μL serum), 21 days after chronic K/BxN serum transfer (single administration of 150 μL serum followed by 2 weekly boosts of 50 μL serum), or from control uninjected mice. FIG. 1A shows expression levels of PTPRS and Cadherin-11 (CAD11) as measured by qPCR. To determine whether expression of PTPRS is elevated in humans, expression levels of PTPRS were assessed by qPCR from normal human dermal fibroblasts and cultured FLS isolated from a rheumatoid joint. The results are shown in FIG. 1B. PTPRS is also elevated in FLS isolated from a rheumatoid join in a human.

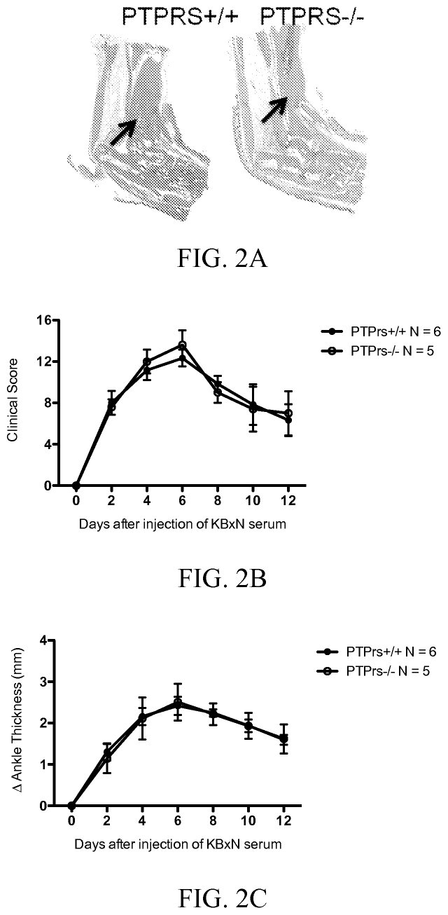

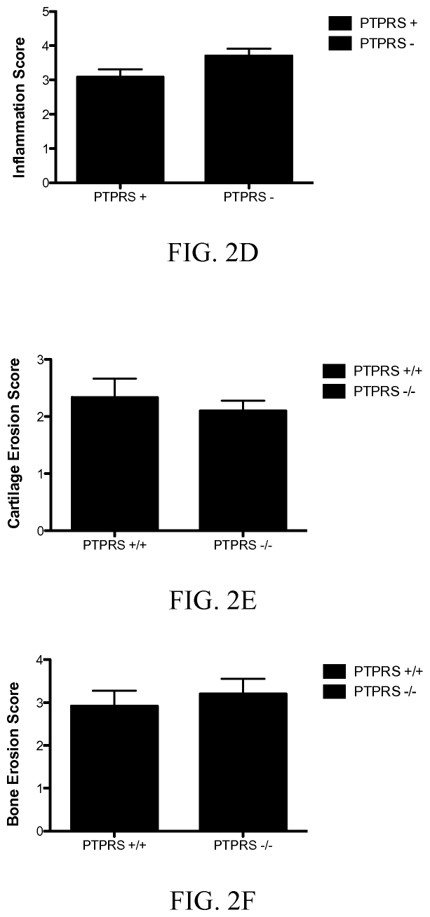

Increased bone erosion severity in arthritis for PTPRS −/− mice as compared with PTPRS +/+ mice was observed. The results are shown in FIGS. 2A-2F. Mice were injected with 100 μL K/BxN serum intraperitoneally at 8-10 weeks of age (PTPRS−/− received proportionally less serum to reflect their decreased body weight). Ankle thickness was measured with digitals calipers and ankles and wrists were given a clinical arthritis score between 0 (no redness or swelling) and 4 (maximal swelling with digits involved) every two days for 14 days. See FIGS. 2A, 2B, and 2C. After 14 days, mice were euthanized and ankles fixed for histological scoring using H&E (inflammation and bone erosion) and safranin O (cartliage erosion) staining. See FIGS. 2D (inflammation), 2E (cartilage erosion), and 2F (bone erosion).

In order to further investigate the role of PTPRS, primary mouse FLS were isolated and evaluated. Knee, ankle and elbow joints were pooled from individual mice and minced in warm RPMI media under sterile conditions. Minced joints from each mouse were incubated in 40 mL warm RPMI media containing 0.5 mg/mL collagenase type IX for 2 hours at room temperature on a rocking platform. Cells and joint pieces were collected by centrifugation and resuspended in FLS medium (DMEM containing 10% FBS, 100 IU/mL penicillin/100 μg/mL streptomycin, 10 μg/mL gentamycin, 2 mM glutamine) and plated in T75 flasks for 3 days before changing media. Once cells reached ˜90% confluence, joint pieces were rinsed away and cells were sub-cultured. Cells were expanded and frozen down at passage 2 and were used for experiments between passages 4 and 8.

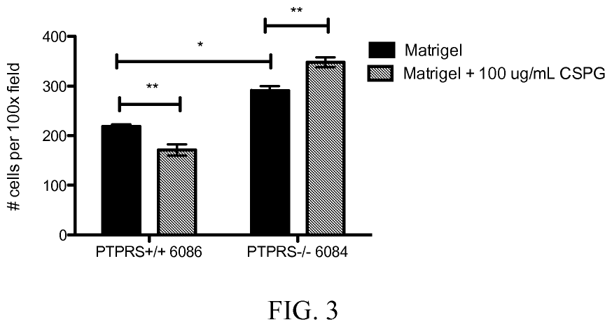

mFLS from PTPRS −/− mice were grown to ˜90% confluence and starved overnight. Transwell inserts (0.8 μm pore size) were coated with 100 μL matrigel diluted 1:25 in cold serum-free media containing no or 100 μg/mL chondroitin sulfate (CS) and incubated at 37° C. for 2 hours before placing in 600 μL FLS media containing 5% FBS. 2.5×10⁴ cells were plated in each transwell chamber in 100 μL FLS media containing 0.5% BSA. Cells were allowed to invade for three days at 37° C., after which transwell inserts were rinsed with PBS and cells on the upper membrane were removed with a cotton swab. Cells that were the bottom membrane surface were fixed with methanol for 10 minutes and stained with crystal violet for 30 minutes. Cells were visualized using a Nikon 80i light microscope and five 100× fields per membrane were used for counting. The results are shown in FIG. 3. The graph is representative of three independent experiments; three wells per treatment; cell lines from female littermates. FIG. 3 shows that PTPRS −/− FLS exhibit reduced CS-mediated inhibition of invasion.

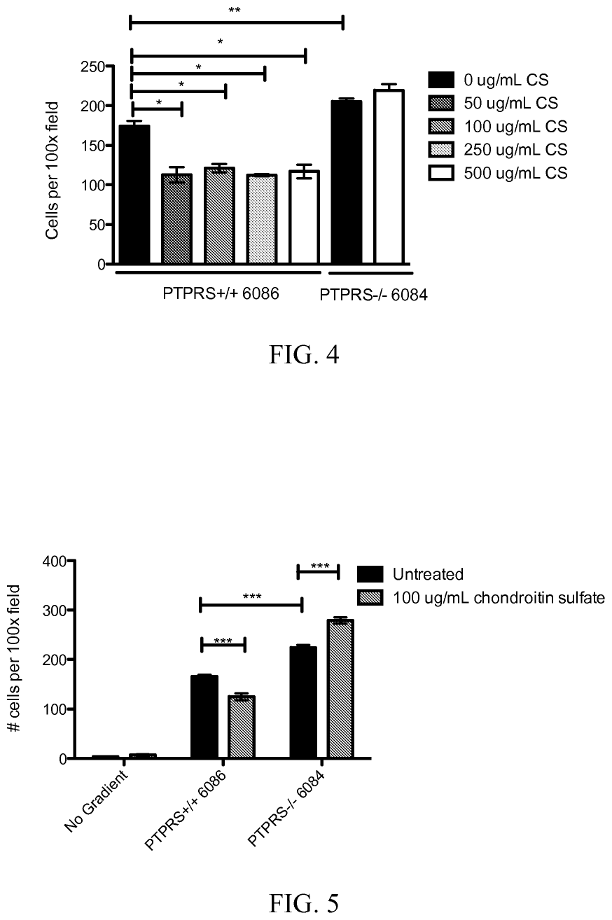

mFLS from PTPRS +/+ and PTPRS −/− mice were grown to ˜90% confluence and starved overnight. Transwell inserts (0.8 μm pore size) were pre-wet with serum-free FLS media at 37° C. for 30 minutes before placing in 600 μL FLS media containing 5% FBS. 2.5×10⁴ cells were plated in each transwell chamber in 100 μL FLS media containing 0.5% BSA and increasing concentrations of chondroitin sulfate (CS). Cells were allowed to migrate for 4 hours at 37° C., after which transwell inserts were rinsed with PBS and cells on the upper membrane were removed with a cotton swab. Cells migrating to the bottom membrane surface were fixed with methanol for 10 minutes and stained with crystal violet for 30 minutes. Cells were visualized using a Nikon 80i light microscope and five 100× fields per membrane were used for counting. The results are shown in FIG. 4. The asterisk signifies statistical significant difference between the two samples. FIG. 4 shows that PTPRS−/− mFLS are insensitive to CS at levels approximately 5 times higher than those in the joint.

mFLS from PTPRS +/+ and PTPRS −/− mice were grown to ˜90% confluence and starved overnight. Transwell inserts (0.8 μm pore size) were pre-wet with serum-free FLS media at 37° C. for 30 minutes before placing in 600 μL FLS media containing 5% FBS. 2.5×10⁴ cells were plated in each transwell chamber in 100 μL FLS media containing 0.5% BSA with or without 100 μg/mL chondroitin sulfate (CS). Cells were allowed to migrate for 4 hours at 37° C., after which transwell inserts were rinsed with PBS and cells on the upper membrane were removed with a cotton swab. Cells migrating to the bottom membrane surface were fixed with methanol for 10 minutes and stained with crystal violet for 30 minutes. Cells were visualized using a Nikon 80i light microscope and five 100× fields per membrane were used for counting. The results are shown in FIG. 5, which are representative of three independent experiments; three wells per treatment; cell lines from female littermates; confirmed in second independent pair of male cell lines. FIG. 5 shows that PTPRS−/− mFLS exhibit reduced CS-mediated inhibition of migration.

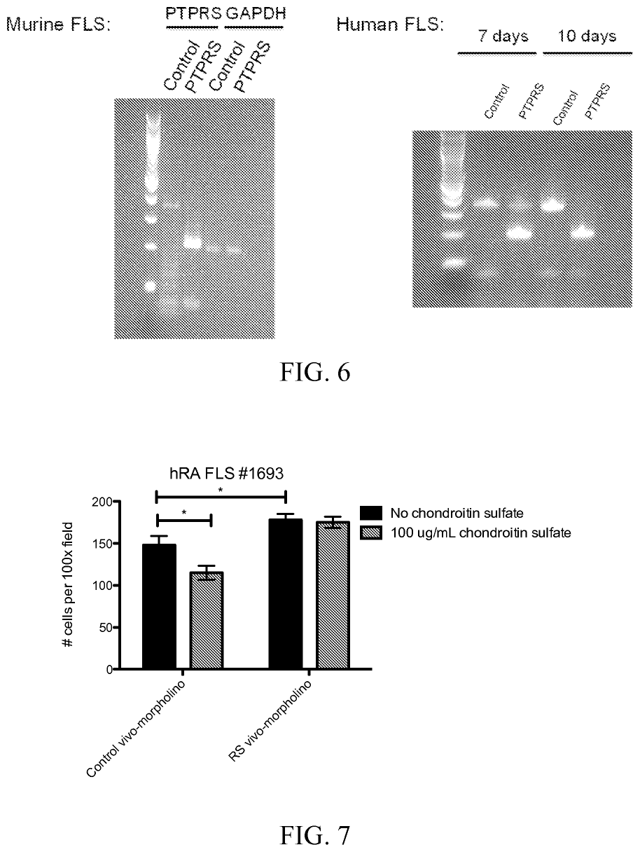

Cell permeable morpholino antisense oligos were obtained from GeneTools (Philomath, Oreg.) and were designed to bind intron-exon junctions and cause the splicing of a selected exon resulting in a frameshift. This ultimately causes a truncated, non-functional mRNA product after 7 days. The results are shown in FIG. 6. The morpholinos mediated knockdown of PTPRS in mouse and human primary FLS.

To determine the effect of PTPRS knockdown in human rheumatoid arthritis (RA), hRA FLS were seeded in 6-well plates and allowed to adhere overnight. Media was replaced containing 2.5 μM morpholino oligo (either control or directed against PTPRS) and knockdown was achieved after 7 days (changing media and oligo at day 3). Cells were then starved overnight prior to migration assay. Transwell inserts (0.8 μm pore size) were pre-wet with serum-free FLS media at 37° C. for 30 minutes before placing in 600 μL FLS media containing 5% FBS. 5×10⁴ cells were plated in each transwell chamber in 100 μL FLS media containing 0.5% BSA with or without 100 μg/mL chondroitin sulfate (CS). Cells were allowed to migrate for 24 hours at 37° C., after which transwell inserts were rinsed with PBS and cells on the upper membrane were removed with a cotton swab. Cells migrating to the bottom membrane surface were fixed with methanol for 10 minutes and stained with crystal violet for 30 minutes. Cells were visualized using a Nikon 80i light microscope and five 100× fields per membrane were used for counting. The results are shown in FIG. 7, which are representative of five independent experiments in three patient cell lines; three wells per treatment. FIG. 7 shows PTPRS knockdown increases migration of human RA FLS and abrogates CS-mediated inhibition of migration.

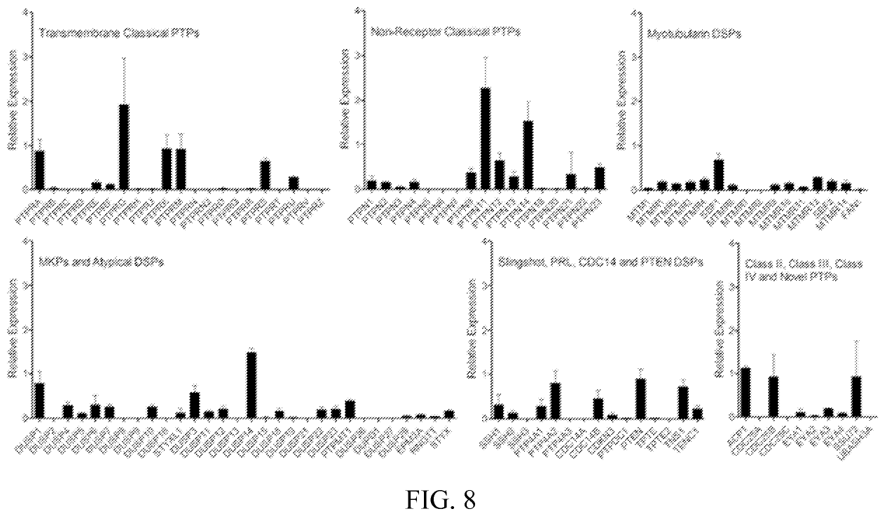

To investigate further the role of PTPs in autoimmune disease, the mRNA PTPome of RA FLS was analyzed. The results are shown in FIG. 8. At least 5 PTPs were highly expressed in RA FLS, one of which was PTPRS.

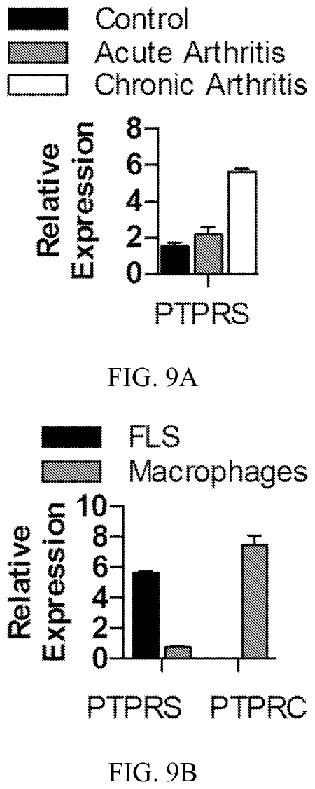

To further study the expression of PTPRS, expression in FLS and macrophages from pooled mice was measured by quantitative polymerase chain reaction. The results are shown in FIGS. 9A and 9B. Values are the mean of PTP expression relative to the housekeeping gene GAPDH. FIG. 9A shows FLS were sorted from control mice or mice with K/BxN serum induced acute or chronic arthritis and FIG. 9B shows mouse sorted macrophages and FLS with PTPRC included as a positive control for expression in macrophages.

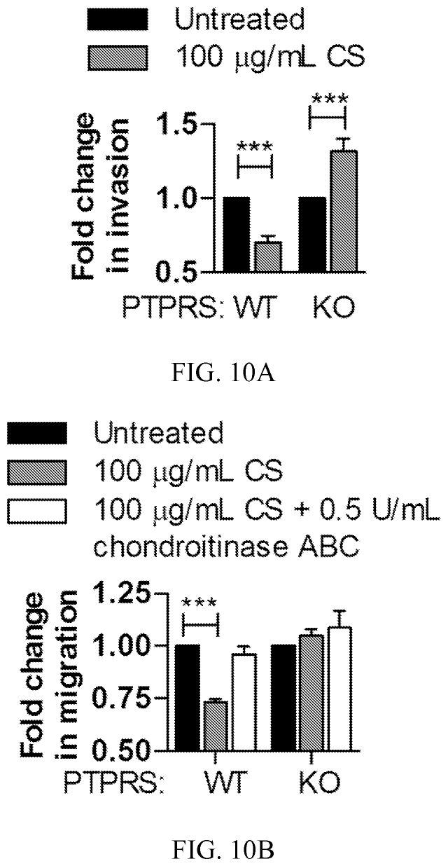

To further study the effects of PTPRS on FLS migration, PTPRS WT or KO FLS were allowed to invade through Matrigel-coated transwell chambers. Cells per field were counted after invasion for 24 hours in response to 5% fetal bovine serum (FBS) in the presence of chondroitin sulfate (CS). The results are shown in FIG. 10A. In FIG. 10B, PTPRS WT or KO FLS were allowed to migrate through uncoated transwell chambers. Cells per field were counted after migration for 4 hours in response to 5% FBS in the presence of CS with or without chondroitinase ABC treatment. Bars indicate mean +/− standard error of the fold-change of migration relative to that of untreated cells. Data include three independent experiments using three sets of littermate cell lines. (***P<0.001; significance determined using Mann-Whitney test on raw data). FIGS. 10A and 10B show that PTPRS KO mice FLS did not migrate in response to CS.

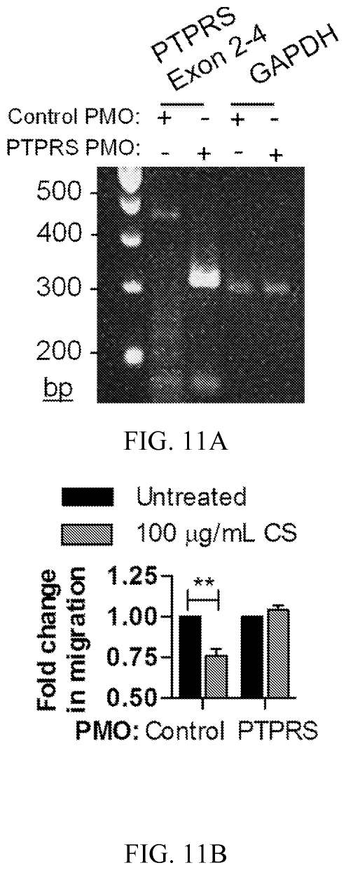

As shown in FIGS. 11A and 11B, the inhibition of RA FLS migration by chondroitin sulfate (CS) is dependent on PTPRS expression. FIG. 11A is a graph of RT-PCR data showing knock-down of PTPRS in RA FLS after incubation with anti-sense oligo (ASO) for 7 days. In FIG. 11B, PTPRS WT or KO FLS were allowed to migrate through uncoated transwell chambers. Cells per field were counted after migration for 4 h in response to 5% fetal bovine serum (FBS) in the presence of CS with or without chondroitinase ABC treatment. Bars indicate mean +/− standard error of the fold-change of migration relative to that of untreated cells. Data include three independent experiments using three sets of littermate cell lines. (***P<0.001; significance determined using Mann-Whitney test on raw data).

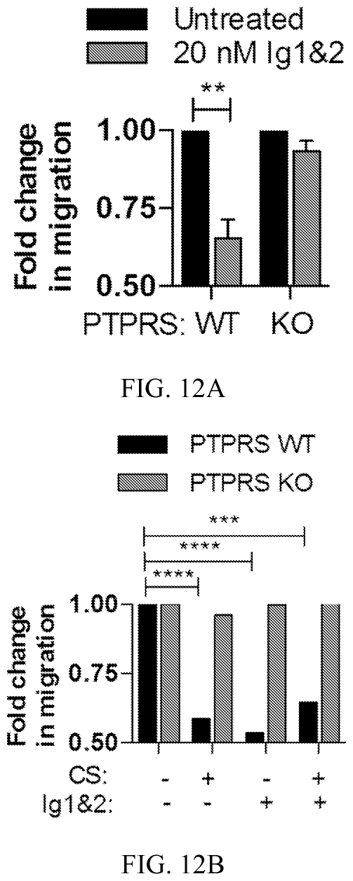

As shown in FIGS. 12A and 12B, displacement of PTPRS from heparin sulfate (HS) is sufficient to inhibit FLS migration and invasion. PTPRS WT or KO FLS were allowed to invade through uncoated transwell chambers. Cells per field were counted after migration for 4 h in response to 5% fetal bovine serum (FBS) in the presence of 20 nM Ig1+2 alone (FIG. 12A) or in addition to 100 μg/mL CS (FIG. 12B). Bars indicate mean +/− standard error of the fold-change of migration relative to that of untreated cells. Data include three independent experiments using three sets of littermate cell lines. (**P<0.01, ***P<0.001, ****P<0.0001; significance determined using Mann-Whitney test on raw data).

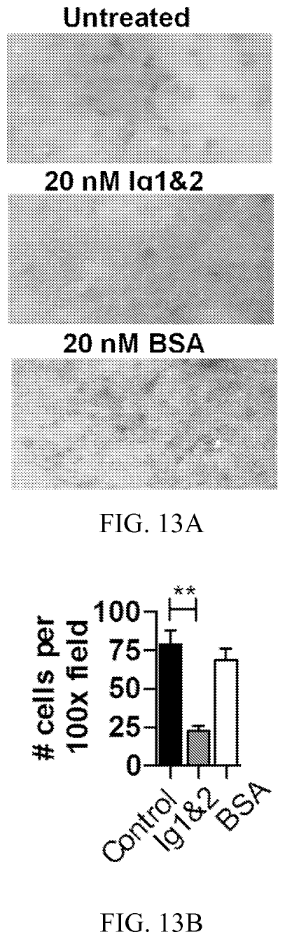

To study the effects of Ig1 and 2 on bovine cartilage, light micrographs of bovine cartilage explants were taken in the presence and absence of Ig1+2 treatment. FIG. 13A shows light micrographs stained with Mayer's hematoxylin with and without Ig1+2 treatments. FIG. 13B is a graph showing cell counts with and without Ig1+2 treatments.

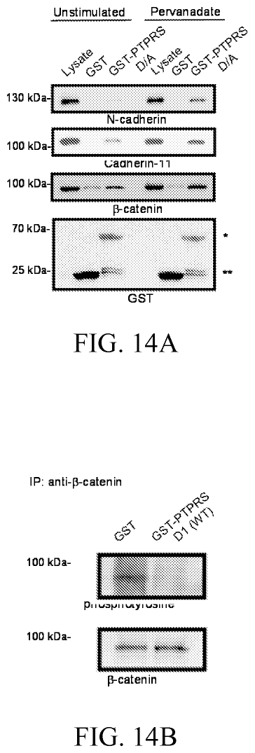

FIGS. 14A and 14B are graphs showing beta-catenin and cadherin binding and desphosphorylation. FIG. 14A is a Western blot of total cell lysates or lysates incubated with GST or GST-PTPRS D/A substrate trapping mutant from unstimulated or pervanadate stimulated FLS. * represents GST-PTPRS D1 D/A and ** represents GST tag. FIG. 14B Western blot showing decreased tyrosine phosphorylation of immunopurified beta-catenin in the presence of GST-PTPRS D1 compared to GST alone.

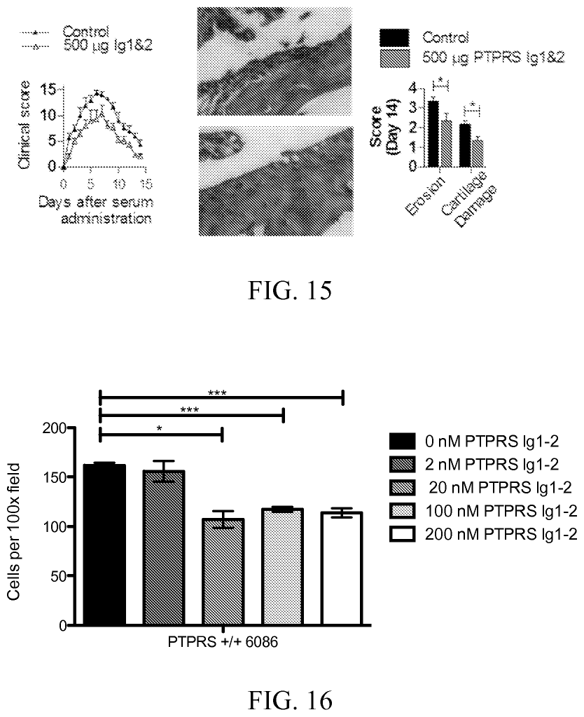

FIG. 15 is a graph showing the effect of Ig1 and Ig2 in ameliorating arthritis. Clinical Score of BALB/c mice induced with acute arthritis by administration of 100 μL K/BxN serum on Day 0 and treated with vehicle (control) or 500 μg Ig1+2 daily from days 0 to 6. N=3 per treatment. Histopathological analysis of the joint with and without Ig1+2 treatment plus erosion and cartilage damage score.

As shown in FIG. 16, treatment of mFLS with recombinant PTPRS Ig1+2 reduces migration. mFLS were grown to ˜90% confluence and starved overnight. Transwell inserts (0.8 μm pore size) were pre-wet with serum-free FLS media at 37° C. for 30 minutes before placing in 600 μL FLS media containing 5% FBS. 2.5×10⁴ cells were plated in each transwell chamber in 100 μL FLS media containing 0.5% BSA with increasing concentrations of recombinant mouse PTPRS Ig1+2 domains. Cells were allowed to migrate for 4 hours at 37° C., after which transwell inserts were rinsed with PBS and cells on the upper membrane were removed with a cotton swab. Cells migrating to the bottom membrane surface were fixed with methanol for 10 minutes and stained with crystal violet for 30 minutes. Cells were visualized using a Nikon 80i light microscope and five 100× fields per membrane were used for counting. FIG. 16 shows that recombinant PTPRS Ig1+2 reduces migration of FLS.

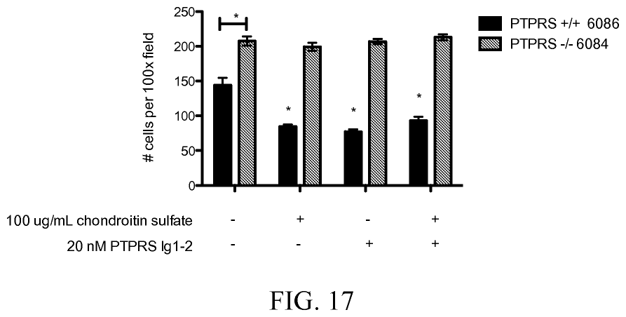

As shown in FIG. 17, PTPRS−/− mFLS are insensitive to recombinant PTPRS Ig1+2 mediated reduced migration. mFLS were grown to ˜90% confluence and starved overnight. Transwell inserts (0.8 μm pore size) were pre-wet with serum-free FLS media at 37° C. for 30 minutes before placing in 600 μL FLS media containing 5% FBS. 2.5×10⁴ cells were plated in each transwell chamber in 100 μL FLS media containing 0.5% BSA and in the presence or absence of 20 nM recombinant mouse PTPRS Ig1+2 domains and/or 100 μg/mL chondroitin sulfate (CS). Cells were allowed to migrate for 4 hours at 37° C., after which transwell inserts were rinsed with PBS and cells on the upper membrane were removed with a cotton swab. Cells migrating to the bottom membrane surface were fixed with methanol for 10 minutes and stained with crystal violet for 30 minutes. Cells were visualized using a Nikon 80i light microscope and five 100× fields per membrane were used for counting. The asterisk in FIG. 17 indicates three wells per treatment and cell lines from female littermates. FIG. 17 shows that PTPRS−/− mFLS do not respond to PTPRS Ig1+2 treatment.

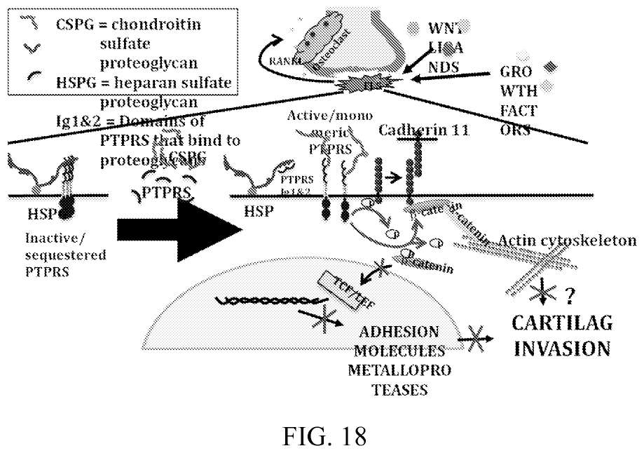

The example and data provided herein demonstrate that PTPRS is highly expressed in FLS and binds chondroitin sulfate (CS) with high affinity. Further, PTPRS mediates the inhibitory action of CS on FLS migration and invasion and recombinant CS-binding domain of PTPRS replicates the action of CS at nanomolar concentrations. This data suggest that proposed model of competition between CS and HS for PTPRS binding is correct. See FIG. 18. This data further suggest that PTPRS is normally mostly in inactive conformation due to heavy binding to HS and that displacement of HS using recombinant Ig1+2 or CS-mimicking antibodies enables activation of the phosphatase. PTPRS is expressed at low levels in hemopoietic cells and KO mouse has no hemopoietic phenotype suggesting anti-PTPRS therapy would be minimally immunosuppressive.

### EMBODIMENTS

Embodiment 1. A non-enzymatic recombinant protein comprising an amino acid sequence of an extracellular domain of PTPRS.

Embodiment 2. The non-enzymatic recombinant protein of embodiment 1, wherein the extracellular domain of PTPRS comprises one or more of PTPRS immunoglobulin-like domain 1 (Ig1), immunoglobulin-like domain 2 (Ig2) or immunoglobulin-like domain 2 (Ig3).

Embodiment 3. The non-enzymatic recombinant protein of embodiment 1 or embodiment 2, wherein the extracellular domain of PTPRS comprises one or both of PTPRS immunoglobulin-like domain 1 (Ig 1) and immunoglobulin-like domain 2 (Ig2).

Embodiment 4. The non-enzymatic recombinant protein of any one of embodiments 1 to 3 wherein the protein comprises Ig1 amino acid residues 30 to 127 of SEQ ID NO:4 or amino acid residues 30 to 127 of SEQ ID NO:8.

Embodiment 5. The non-enzymatic recombinant protein of embodiment 4, wherein the protein comprises an amino acid sequence set forth as: EEPRFIKEPKDQIGVSGGVASFVCQATGDPKPRVTWNKKGKKVNSQRFETIEFDESAGA VLRIQPLRTPRDENVYECVAQNSVGEITVHAKLTVLRE(SEQ ID NO:1) or as set forth in SEQ ID NO:5.

Embodiment 6. The non-enzymatic recombinant protein of any one of embodiments 1 to 3, wherein the protein comprises Ig2 amino acid residues 128 to 231 of SEQ ID NO:4 or amino acid residues 128-244 of SEQ ID NO:8.

Embodiment 7. The non-enzymatic recombinant protein of embodiment 6, wherein the protein comprises an amino acid sequence set forth as: DQLPSGFPNIDMGPQLKVVERTRTATMLCAASGNPDPEITWFKDFLPVDPSASNGRIKQL RSETFESTPIRGALQIESSEETDQGKYECVATNSAGVRYSSPANLYVRVRRVA (SEQ ID NO:2) or as set forth in SEQ ID NO:6.

Embodiment 8. The non-enzymatic recombinant protein of embodiment 1 or embodiment 2, wherein the protein comprises Ig3 amino acid residues 232-321 of SEQ ID NO:4 or amino acid residues of 245-334 of SEQ ID NO:8.

Embodiment 9. The non-enzymatic recombinant protein of embodiment 8, wherein the protein comprises an amino acid sequence set forth as: PRFSILPMSHEIMPGGNVNITCVAVGSPMPYVKWMQGAEDLTPEDDMPVGRNVLELTD VKDSANYTCVAMSSLGVIEAVAQITVKSLPKA (SEQ ID NO:3) or as set forth in SEQ ID NO:7.

Embodiment 10. The non-enzymatic recombinant protein of any one of embodiments 1 to 9, wherein the protein binds heparan sulfate.

Embodiment 11. The non-enzymatic recombinant protein of any one of embodiments 1 to 10, wherein the protein lacks a transmembrane domain.

Embodiment 12. The non-enzymatic recombinant protein of any one of embodiments 1 to 11, wherein the protein lacks an intracellular domain.

Embodiment 13. A pharmaceutical composition comprising the non-enzymatic recombinant protein of any one of embodiments 1 to 12 and a pharmaceutically acceptable excipient.

Embodiment 14. A kit comprising the non-enzymatic recombinant protein of any one of embodiments 1 to 12 and instructions for use.

Embodiment 15. The non-enzymatic recombinant protein of any one of embodiments 1 to 12 for use in the treatment of a subject who has or is at risk of developing an autoimmune disease.

Embodiment 16. The non-enzymatic recombinant protein of any one of embodiments 1 to 12, for use in the treatment of a subject who has or is at risk of developing arthritis.

Embodiment 17. The non-enzymatic recombinant protein of any one of embodiments 1 to 12, for use in the treatment of a subject who has or is at risk of developing an extracellular matrix disease.

Embodiment 18. The non-enzymatic protein of any one of embodiments 1 to 12 for use in the treatment of a subject who has or is at risk of developing a fibroblast-mediated disease.

Embodiment 19. A method of treating an autoimmune disease in a subject, the method comprising administering to the subject a therapeutically effective amount of a PTPRS de-clustering agent, wherein administration treats the autoimmune disease in the subject, and wherein the de-clustering agent is not chondroitin sulfate.

Embodiment 20. A method of decreasing fibroblast activity in a subject, the method comprising administering to the subject a therapeutically effective amount of a PTPRS de-clustering agent, wherein administration decreases fibroblast activity in the subject, and wherein the de-clustering agent is not chondroitin sulfate.

Embodiment 21. The method of embodiment 19 or 20, wherein the PTPRS de-clustering agent is the non-enzymatic recombinant protein of any one of 1 to 12.

Embodiment 22. The method of embodiment 19 or 20, wherein the PTPRS de-clustering agent is an anti-PTPRS antibody or fragment thereof.

Embodiment 23. The method of embodiment 19 or 20, wherein the PTPRS de-clustering agent binds heparan sulfate.

Embodiment 24. The method of embodiment 19 or 20, wherein the PTPRS de-clustering agent is an anti-heparan sulfate antibody.

Embodiment 25. The method of any one of embodiments 19 or 21 to 24, wherein the autoimmune disease is arthritis.

Embodiment 26. The method of any one of embodiments 19 or 21 to 24, wherein the autoimmune disease is rheumatoid arthritis.

Embodiment 27. The method of any one of embodiments 19 or 21 to 24, wherein the autoimmune disease is scleroderma or Crohn's disease.

Embodiment 28. The method of any one of embodiments 20 to 24, wherein the fibroblast activity comprises fibroblast migration.

Embodiment 29. The method of any one of embodiments 20 to 24, wherein the fibroblast activity comprises collagen production, glycosaminoglycan production, reticular and elastic fiber production, cytokine production, chemokine production, glycoprotein production or combinations thereof.

Embodiment 30. The method of any one of embodiments 20 to 24, wherein the fibroblast activity comprises extracellular matrix production.

Embodiment 31. The method of any one of embodiments 20 to 24, wherein the fibroblast is selected from the group consisting of synovial fibroblasts, dermal fibroblasts, and interstitial fibroblasts.

Embodiment 32 . The method of embodiment 31, wherein the fibroblasts are synovial fibroblasts.

Embodiment 33. The method of any one of embodiments 20 to 24, wherein the subject has a fibroblast-mediated disease.

Embodiment 34. The method of embodiment 33, wherein the fibroblast-mediated disease is fibrosis.

Embodiment 35. The method of embodiment 34, wherein the fibrosis is pulmonary fibrosis, idiopathic pulmonary fibrosis, liver fibrosis, endomyocardial fibrosis, atrial fibrosis, mediastinal fibrosis, myelofibrosis, retroperitoneal fibrosis, nephrogenic systemic fibrosis, skin fibrosis, or arthrofibrosis.

Embodiment 36 . The method of embodiment 33, wherein the fibroblast-mediated disease is a fibroblast-mediated autoimmune disease.

Embodiment 37. The method of embodiment 36, wherein the fibroblast-mediated autoimmune disease is selected from the group consisting of Crohn's disease, arthritis, rheumatoid arthritis, and scleroderma.

Embodiment 38. A method of treating a fibroblast mediated disease in a subject, the method comprising administering to the subject a therapeutically effective amount of a PTPRS de-clustering agent, wherein administration treats the fibroblast-mediated disease in the subject, and wherein the de-clustering agent is not chondroitin sulfate.

Embodiment 39. The method of embodiment 38, wherein the PTPRS de-clustering agent is the non-enzymatic recombinant protein of any one of 1 to 12.

Embodiment 40. The method of embodiment 38, wherein the PTPRS de-clustering agent is an anti-PTPRS antibody or fragment thereof.

Embodiment 41. The method of embodiment 38, wherein the PTPRS de-clustering agent binds heparan sulfate.

Embodiment 42. The method of embodiment 38, wherein the PTPRS de-clustering agent is an anti-heparan sulfate antibody.

Embodiment 43. The method of any one of embodiments 38 to 42, wherein the fibroblast-mediated disease is fibrosis.

Embodiment 44. The method of embodiment 43, wherein the fibrosis is pulmonary fibrosis, idiopathic pulmonary fibrosis, liver fibrosis, endomyocardial fibrosis, atrial fibrosis, mediastinal fibrosis, myelofibrosis, retroperitoneal fibrosis, nephrogenic systemic fibrosis, skin fibrosis, or arthrofibrosis.

Embodiment 45. The method of any one of embodiments 38 to 42, wherein the fibroblast-mediated disease is a fibroblast-mediated autoimmune disease.

Embodiment 46. The method of embodiment 45, wherein the fibroblast-mediated autoimmune disease is selected from the group consisting of Crohn's disease, arthritis, rheumatoid arthritis, and scleroderma.

Embodiment 47. A method of modulating extracellular matrix in a subject, the method comprising administering to the subject an effective amount of the non-enzymatic recombinant protein of any one of embodiments 1 to 12, wherein administration modulates the extracellular matrix in the subject.

Embodiment 48 . The method of embodiment 47, wherein modulation of the extracellular matrix comprises modulation of one or more components of the extracellular matrix.

Embodiment 49. The method of embodiment 48, wherein the extracellular matrix component is selected from the group consisting of a proteoglycan, polysaccharide or fiber.

Embodiment 50. The method of embodiment 49, wherein the extracellular matrix component is a proteoglycan.

Embodiment 51. The method of embodiment 50, wherein the proteoglycan is heparan sulfate.

Embodiment 52. The method of any one of embodiments 47 to 51, wherein the subject has an extracellular matrix disease.

Embodiment 53. The method of embodiment 52, wherein the extracellular matrix disease is selected from the group consisting of atherosclerosis, cancer, an amyloid disease, an inflammatory condition, and a developmental disorder.

Embodiment 54. The method of embodiment 53, wherein the amyloid disease is Alzheimer's disease or inflammation-related AA amyloidosis.

Embodiment 55. The method of embodiment 53, wherein the inflammatory condition is osteoarthritis, systemic scleroderma, or lupus.

Embodiment 56. A method of identifying a candidate PTPRS de-clustering agent, the method comprising contacting a test agent with clustered PTPRS peptides and detecting de-clustering of the PTPRS peptides, thereby identifying a candidate PTPRS de-clustering agent.

Embodiment 57. A method of identifying a candidate PTPRS de-clustering agent, the method comprises contacting a test agent with PTPRS and heparan sulfate and determining whether the test agent inhibits binding of the PTPRS to heparan sulfate, inhibition of binding indicating the test agent is a PTPRS de-clustering agent.

Embodiment 58. The method of embodiment 56 or 57, wherein the test agent is a nucleic acid, peptide, antibody or small molecule.

Embodiment 59. A method of determining whether a subject has or is at risk for developing an autoimmune disease comprising obtaining a biological sample from the subject and determining whether the subject has an altered RNA level or an altered protein level of an PTPRS, an elevated altered RNA level or elevated protein level indicating the subject has or is at risk of developing an autoimmune disease.

Embodiment 60. A method of detecting a level of PTPRS RNA or PTPRS protein in a subject that has or is suspected of having an autoimmune disease, the method comprising obtaining a biological sample from the subject and measuring a level of PTPRS RNA or PTRPRS protein in the biological sample.

Embodiment 61. The method of embodiment 59 or embodiment 60, further comprising designating the subject for autoimmune disease therapy.

Embodiment 62. The method of embodiment 59 or embodiment 60, further comprising administering to the subject an autoimmune disease therapy.

Embodiment 63. The method of embodiment 59 or embodiment 60, further comprising selecting a subject with an autoimmune disease.

Embodiment 64. The method of any one of embodiments 59 to 63, wherein the autoimmune disease is selected from the group consisting of Crohn's disease, arthritis, rheumatoid arthritis, and or scleroderma.

Embodiment 65. The method of embodiment 64, wherein the autoimmune disease is rheumatoid arthritis.

Embodiment 66. The method of any one of embodiments 59 to 65, wherein the biological sample is derived from a joint tissue or bodily fluid.

Embodiment 67. The method of embodiment 66, further comprising isolating cells from the joint tissue or bodily fluid thereby forming an isolated cell sample.

Embodiment 68. The method of embodiment 67, wherein the isolated cell sample comprises synoviocytes, fibroblasts, hematopoetic cells, macrophages, leukocytes, T-cells or a combination thereof.

Embodiment 69. The method of embodiment 68, wherein the synoviocytes are fibroblast-like synoviocytes or macrophage-like synoviocytes.

Embodiment 70. The method of embodiment 67, wherein the isolated cell sample comprises fibroblast-like synoviocytes.

Embodiment 71. The method of embodiment 67, wherein the bodily fluid is whole blood, plasma, serum, urine, sputum, saliva, a bronchioaviolar lavage sample, or synovial fluid.

Embodiment 72. The method of embodiment 67, wherein the bodily fluid is synovial fluid.

Embodiment 73. A compound comprising a protein or peptide comprising, consisting of or consisting essentially of an amino acid sequence of an extracellular domain of PTPRS, or a subsequence, portion, homologue, variant or derivative thereof.

Embodiment 74. The compound of embodiment 73 wherein the extracellular domain of PTPRS comprises one or more of PTPRS immunoglobulin-like domain 1 (Ig1), immunoglobulin-like domain 2 (Ig2) and immunoglobulin-like domain 2 (Ig3), or a subsequence, portion, homologue, variant or derivative thereof.

Embodiment 75. The compound of embodiment 73 or embodiment 74 wherein the extracellular domain of PTPRS comprises one or both of PTPRS immunoglobulin-like domain 1 (Ig1) and immunoglobulin-like domain 2 (Ig2) or a subsequence, portion, homologue, variant or derivative thereof.

Embodiment 76. The compound of any one of embodiments 73 to 75 wherein the compound comprises a protein or peptide comprising, consisting of or consisting essentially of Ig1 amino acid residues 39 to 124, or a subsequence, portion, homologue, variant or derivative thereof.

Embodiment 77. The compound of embodiment 76 wherein the compound comprises a protein or peptide comprising, consisting of or consisting essentially of an amino acid sequence set forth as: PKDQIGVSGGVASFVCQATGDPKPRVTWNKKGKKVNSQRFETIEFDESAGAVLRIQPLR TPRDENVYECVAQNSVGEITVHAKLTV, or a subsequence, portion, homologue, variant or derivative thereof.

Embodiment 78. The compound of any one of embodiments 73 to 75 wherein the compound comprises a protein or peptide comprising, consisting of or consisting essentially of Ig2 amino acid residues 152 to 233, or a subsequence, portion, homologue, variant or derivative thereof.

Embodiment 79. The compound of embodiment 78 wherein the compound comprises a protein or peptide comprising, consisting of or consisting essentially of an amino acid sequence set forth as: ATMLCAASGNPDPEITWFKDFLPVDPSASNGRIKQLRSETFESTPIRGALQIESSEETDQG KYECVATNSAGVRYSSPANLY, or a subsequence, portion, homologue, variant or derivative thereof.

Embodiment 80. The compound of embodiment 73 or embodiment 74 wherein the compound comprises a protein or peptide comprising, consisting of or consisting essentially of Ig3 amino acid residues 259-327, or a subsequence, portion, homologue, variant or derivative thereof.

Embodiment 81. The compound of embodiment 80 wherein the compound comprises a protein or peptide comprising, consisting of or consisting essentially of an amino acid sequence set forth as: PRFSILPMSHEIMPGGNVNITCVAVGSPMPYVKWMQGAEDLTPEDDMPVGRNVLELTD VKDSANYTCVAMSSLGVIEAVAQITVKSLPKA, or a subsequence, portion, homologue, variant or derivative thereof.

Embodiment 82. The compound of any one of embodiments 73 to 81 wherein the protein or peptide binds heparan sulphate proteoglycan.

Embodiment 83. The compound of any one of embodiments 73 to 82 wherein the compound prevents oligomerization or clustering of PTPRS proteins.

Embodiment 84. The compound of embodiment 83 wherein the compound prevents dimerization of PTPRS proteins.

Embodiment 85. The compound of any one of embodiments 73 to 84 wherein the compound modulates PTPRS activity.

Embodiment 86. The compound of any one of embodiments 73 to 85 wherein the compound increases phosphatase activity of PTPRS.

Embodiment 87. The compound of any one of embodiments 73 to 86 for use in the treatment of a subject who has or is at risk of developing an autoimmune disease.

Embodiment 88. The compound of any one of embodiments 73 to 87 for use in the treatment of a subject who has or is at risk of developing arthritis.

Embodiment 89. The compound of any one of embodiments 73 to 88 for use in the treatment of a subject who has or is at risk of developing rheumatoid arthritis.

Embodiment 90. A method of modulating PTPRS activity in a subject, the method comprising administering to the subject a compound that decreases, reduces, inhibits, suppresses or disrupts oligomerization or clustering of PTPRS proteins.

Embodiment 91. A method of treating a subject for an autoimmune disease, the method comprising administering to the subject a compound that decreases, reduces, inhibits, suppresses or disrupts oligomerization or clustering of PTPRS proteins.

Embodiment 92. A method of treating a subject for an autoimmune disease, the method comprising administering to the subject a compound that increases PTPRS phosphatase activity.

Embodiment 93. The method of any one of embodiments 90 to 92 comprising administering to the subject the compound of any one of 73 to 89.

Embodiment 94. A method of determining whether a subject has or is at risk of developing an autoimmune disease, said method comprising determining whether a subject expresses an elevated RNA level of an RPTPS or an elevated protein level of an RPTPS relative to a standard control, wherein the presence of said elevated RNA level or said elevated protein level indicates said subject has or is at risk of developing an autoimmune disease.

Embodiment 95. The method of embodiment 94, further comprising administering a treatment for said autoimmune disease, wherein said treatment comprises a PTPRS dedimerizing agent.

Embodiment 96. The method of embodiment 95, wherein said PTPRS de-dimerizing agent is not chondroitin.

Embodiment 97. The method of embodiment 95, wherein said PTPRS de-dimerizing agent is an anti-PTPRS antibody or a fragment thereof or a chondroitin mimetic.

Embodiment 98. A method of treating a subject who has or is at risk of developing an autoimmune disease, said method comprising administering to said subject a therapeutically effective amount of a PTPRS de-dimerizing agent.

Embodiment 99. The method of embodiment 98, wherein said PTPRS de-dimerizing agent is not chondroitin.

Embodiment 100. The method of embodiment 98, wherein said PTPRS de-dimerizing agent is an anti-PTPRS antibody or a fragment thereof or a chondroitin mimetic.

Embodiment 101. The method of embodiment 98, wherein said autoimmune disease is an inflammatory autoimmune disease.

Embodiment 102. The method of embodiment 101, wherein said method further comprises, prior to said administering, determining whether a subject expresses an elevated RNA level of said PTPRS or an elevated protein level of said PTPRS, relative to a standard control, wherein the presence of said elevated RNA level or said elevated protein level indicates said subject has or is at risk of developing said inflammatory autoimmune disease.

Embodiment 103. The method of embodiment 102, wherein said determining comprises obtaining a biological sample from said subject, wherein said biological sample is derived from a joint tissue or a bodily fluid.

Embodiment 104. The method of embodiment 103, further comprising isolating cells from said joint tissue or said bodily fluid thereby forming isolated sample cells.

Embodiment 105. The method of embodiment 104, wherein said isolated sample cells are synoviocytes, fibroblast-like synoviocytes, macrophage-like synoviocytes, fibroblasts, hematopoetic cells, macrophages, leukocytes, T cells, or other immune cells.

Embodiment 106. The method of embodiment 104, wherein said isolated cells are fibroblast-like synoviocytes.

Embodiment 107. The method of embodiment 104, wherein said bodily fluid is whole blood, plasma, serum, urine, sputum, saliva, a bronchioaviolar lavage sample, synovial fluid, or exhaled breath condensate.

Embodiment 108. The method of embodiment 104, wherein said bodily fluid is synovial fluid.

Embodiment 109. The method of one of embodiments 101 or 102, wherein said inflammatory autoimmune disease is an arthritis.

Embodiment 110. The method of one of embodiments 101 or 102, wherein said inflammatory autoimmune disease is arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis,

Hashimoto's thyroiditis, ankylosing spondylitis, psoriasis, Sjogren's syndrome,vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, or allergic asthma.

Embodiment 111. The method of one of embodiments 101 or 102, wherein said inflammatory autoimmune disease is rheumatoid arthritis.

Embodiment 112. A method of identifying a candidate RPTPS de-dimerizing agent, said method comprising (i) contacting a test agent with a dimerizered RPTPS; and (ii) detecting de-dimeriziation of said RPTPS, thereby identifying a candidate RPTPS de-dimerization agent.

Embodiment 113. A pharmaceutical composition comprising a PTPRS de-dimerizing agent and a pharmaceutically acceptable excipient.

Embodiment 114. The pharmaceutical composition of embodiment 113, wherein said PTPRS dedimerizing agent is not chondroitin.

Embodiment 115. The pharmaceutical composition of embodiment 113, wherein said PTPRS dedimerizing agent is an anti-PTPRS antibody or a fragment thereof or a chondroitin mimetic.

Embodiment 116. The pharmaceutical composition of embodiment 113, wherein said pharmaceutical composition is formulated for delivery to synovial fluid.

Embodiment 117. The pharmaceutical composition of embodiment 113, wherein said pharmaceutical composition is formulated for delivery to fibroblast-like synoviocytes.

What is claimed is:

1. A non-enzymatic recombinant protein comprising an amino acid sequence of an extracellular domain of PTPRS.

2. The non-enzymatic recombinant protein of claim 1, wherein the extracellular domain of PTPRS comprises one or more of PTPRS immunoglobulin-like domain 1 (Ig1), immunoglobulin-like domain 2 (Ig2) or immunoglobulin-like domain 2 (Ig3).

3. The non-enzymatic recombinant protein of claim 1 or claim 2, wherein the extracellular domain of PTPRS comprises one or both of PTPRS immunoglobulin-like domain 1 (Ig1) and immunoglobulin-like domain 2 (Ig2).

4. The non-enzymatic recombinant protein of any one of claims 1 to 3 wherein the protein comprises Ig1 amino acid residues 30 to 127 of SEQ ID NO:4 or amino acid residues 30-127 of SEQ ID NO:8.

5. The non-enzymatic recombinant protein of claim 4, wherein the protein comprises an amino acid sequence set forth as: EEPRFIKEPKDQIGVSGGVASFVCQATGDPKPRVTWNKKGKKVNSQRFETIEFDESAGA VLRIQPLRTPRDENVYECVAQNSVGEITVHAKLTVLRE(SEQ ID NO:1) or as set forth in SEQ ID NO:5.

6. The non-enzymatic recombinant protein of any one of claims 1 to 3, wherein the protein comprises Ig2 amino acid residues 128 to 231 of SEQ ID NO:4 or amino acid residues 128-244 of SEQ ID NO:8.

7. The non-enzymatic recombinant protein of claim 6, wherein the protein comprises an amino acid sequence set forth as: DQLPSGFPNIDMGPQLKVVERTRTATMLCAASGNPDPEITWFKDFLPVDPSASNGRIKQL RSETFESTPIRGALQIESSEETDQGKYECVATNSAGVRYSSPANLYVRVRRVA (SEQ ID NO:2) or as set forth in SEQ ID NO:6.

8. The non-enzymatic recombinant protein of claim 1 or claim 2, wherein the protein comprises Ig3 amino acid residues 232-321 of SEQ ID NO:4 or amino acid residues 245-334 of SEQ ID NO:8.

9. The non-enzymatic recombinant protein of claim 8, wherein the protein comprises an amino acid sequence set forth as: PRFSILPMSHEIMPGGNVNITCVAVGSPMPYVKWMQGAEDLTPEDDMPVGRNVLELTD VKDSANYTCVAMSSLGVIEAVAQITVKSLPKA (SEQ ID NO:3) or as set forth in SEQ ID NO:7.

10. The non-enzymatic recombinant protein of any one of claims 1 to 9, wherein the protein binds heparan sulfate.

11. The non-enzymatic recombinant protein of any one of claims 1 to 10, wherein the protein lacks a transmembrane domain.

12. The non-enzymatic recombinant protein of any one of claims 1 to 11, wherein the protein lacks an intracellular domain.

13. A pharmaceutical composition comprising the non-enzymatic recombinant protein of any one of claims 1 to 12 and a pharmaceutically acceptable excipient.

14. A kit comprising the non-enzymatic recombinant protein of any one of claims 1 to 12 and instructions for use.

15. The non-enzymatic recombinant protein of any one of claims 1 to 12 for use in the treatment of a subject who has or is at risk of developing an autoimmune disease.

16. The non-enzymatic recombinant protein of any one of claims 1 to 12, for use in the treatment of a subject who has or is at risk of developing arthritis.

17. The non-enzymatic recombinant protein of any one of claims 1 to 12, for use in the treatment of a subject who has or is at risk of developing an extracellular matrix disease.

18. The non-enzymatic protein of any one of claims 1 to 12 for use in the treatment of a subject who has or is at risk of developing a fibroblast-mediated disease.

19. A method of treating an autoimmune disease in a subject, the method comprising administering to the subject a therapeutically effective amount of a PTPRS de-clustering agent, wherein administration treats the autoimmune disease in the subject, and wherein the de-clustering agent is not chondroitin sulfate.

20. A method of decreasing fibroblast activity in a subject, the method comprising administering to the subject a therapeutically effective amount of a PTPRS de-clustering agent, wherein administration decreases fibroblast activity in the subject, and wherein the de-clustering agent is not chondroitin sulfate.

21. The method of claim 19 or 20, wherein the PTPRS de-clustering agent is the non-enzymatic recombinant protein of any one of 1 to 12.

22. The method of claim 19 or 20, wherein the PTPRS de-clustering agent is an anti-PTPRS antibody or fragment thereof.

23. The method of claim 19 or 20, wherein the PTPRS de-clustering agent binds heparan sulfate.

24. The method of claim 19 or 20, wherein the PTPRS de-clustering agent is an anti-heparan sulfate antibody.

25. The method of any one of claims 19 or 21 to 24, wherein the autoimmune disease is arthritis.

26. The method of any one of claims 19 or 21 to 24, wherein the autoimmune disease is rheumatoid arthritis.

27. The method of any one of claims 19 or 21 to 24, wherein the autoimmune disease is scleroderma or Crohn's disease.

28. The method of any one of claims 20 to 24, wherein the fibroblast activity comprises fibroblast migration.

29. The method of any one of claims 20 to 24, wherein the fibroblast activity comprises collagen production, glycosaminoglycan production, reticular and elastic fiber production, cytokine production, chemokine production, glycoprotein production or combinations thereof.

30. The method of any one of claims 20 to 24, wherein the fibroblast activity comprises extracellular matrix production.

31. The method of any one of claims 20 to 24, wherein the fibroblast is selected from the group consisting of synovial fibroblasts, dermal fibroblasts, and interstitial fibroblasts.

32. The method of claim 31, wherein the fibroblasts are synovial fibroblasts.

33. The method of any one of claims 20 to 24, wherein the subject has a fibroblast-mediated disease.

34. The method of claim 33, wherein the fibroblast-mediated disease is fibrosis.

35. The method of claim 34, wherein the fibrosis is pulmonary fibrosis, idiopathic pulmonary fibrosis, liver fibrosis, endomyocardial fibrosis, atrial fibrosis, mediastinal fibrosis, myelofibrosis, retroperitoneal fibrosis, nephrogenic systemic fibrosis, skin fibrosis, or arthrofibrosis.

36. The method of claim 33, wherein the fibroblast-mediated disease is a fibroblast-mediated autoimmune disease.

37. The method of claim 36, wherein the fibroblast-mediated autoimmune disease is selected from the group consisting of Crohn's disease, arthritis, rheumatoid arthritis, and scleroderma.

38. A method of treating a fibroblast mediated disease in a subject, the method comprising administering to the subject a therapeutically effective amount of a PTPRS de-clustering agent, wherein administration treats the fibroblast-mediated disease in the subject, and wherein the de-clustering agent is not chondroitin sulfate.

39. The method of claim 38, wherein the PTPRS de-clustering agent is the non-enzymatic recombinant protein of any one of 1 to 12.

40. The method of claim 38, wherein the PTPRS de-clustering agent is an anti-PTPRS antibody or fragment thereof.

41. The method of claim 38, wherein the PTPRS de-clustering agent binds heparan sulfate.

42. The method of claim 38, wherein the PTPRS de-clustering agent is an anti-heparan sulfate antibody.

43. The method of any one of claims 38 to 42, wherein the fibroblast-mediated disease is fibrosis.

44. The method of claim 43, wherein the fibrosis is pulmonary fibrosis, idiopathic pulmonary fibrosis, liver fibrosis, endomyocardial fibrosis, atrial fibrosis, mediastinal fibrosis, myelofibrosis, retroperitoneal fibrosis, nephrogenic systemic fibrosis, skin fibrosis, or arthrofibrosis.

45. The method of any one of claims 38 to 42, wherein the fibroblast-mediated disease is a fibroblast-mediated autoimmune disease.

46. The method of claim 45, wherein the fibroblast-mediated autoimmune disease is selected from the group consisting of Crohn's disease, arthritis, rheumatoid arthritis, and scleroderma.

47. A method of modulating extracellular matrix in a subject, the method comprising administering to the subject an effective amount of the non-enzymatic recombinant protein of any one of claims 1 to 12, wherein administration modulates the extracellular matrix in the subject.

48. The method of claim 47, wherein modulation of the extracellular matrix comprises modulation of one or more components of the extracellular matrix.

49. The method of claim 48, wherein the extracellular matrix component is selected from the group consisting of a proteoglycan, polysaccharide or fiber.

50. The method of claim 49, wherein the extracellular matrix component is a proteoglycan.

51. The method of claim 50, wherein the proteoglycan is heparan sulfate.

52. The method of any one of claims 47 to 51, wherein the subject has an extracellular matrix disease.

53. The method of claim 52, wherein the extracellular matrix disease is selected from the group consisting of atherosclerosis, cancer, an amyloid disease, an inflammatory condition, and a developmental disorder.

54. The method of claim 53, wherein the amyloid disease is Alzheimer's disease or inflammation-related AA amyloidosis.

55. The method of claim 53, wherein the inflammatory condition is osteoarthritis, systemic scleroderma, or lupus.

56. A method of identifying a candidate PTPRS de-clustering agent, the method comprising contacting a test agent with clustered PTPRS peptides and detecting de-clustering of the PTPRS peptides, thereby identifying a candidate PTPRS de-clustering agent.

57. A method of identifying a candidate PTPRS de-clustering agent, the method comprises contacting a test agent with PTPRS and heparan sulfate and determining whether the test agent inhibits binding of the PTPRS to heparan sulfate, inhibition of binding indicating the test agent is a PTPRS de-clustering agent.

58. The method of claim 56 or 57, wherein the test agent is a nucleic acid, peptide, antibody or small molecule.


SEQUENCE LISTING (13 sequences)

SEQ ID NO: 1 - protein, 97 aa, Artificial Sequence
  Synthetic Construct
EEPRFIKEPKDQIGVSGGVASFVCQATGDPKPRVTWNKKGKKVNSQRFETIEFDESAGAVLRIQPLRTPRDENVYECVAQNSVGEITVHAKLTVLRE

SEQ ID NO: 2 - protein, 113 aa, Artificial Sequence
  Synthetic Construct
DQLPSGFPNIDMGPQLKVVERTRTATMLCAASGNPDPEITWFKDFLPVDPSASNGRIKQLRSETFESTPIRGALQIESSEETDQGKYECVATNSAGVRYSSPANLYVRVRRVA

SEQ ID NO: 3 - protein, 90 aa, Mus musculus
PRFSILPMSHEIMPGGNVNITCVAVGSPMPYVKWMQGAEDLTPEDDMPVGRNVLELTDVKDSANYTCVAMSSLGVIEAVAQITVKSLPKA

SEQ ID NO: 4 - protein, 1907 aa, Mus musculus
MAPTWSPSVVSVVGPVGLFLVLLARGCLAEEPPRFIREPKDQIGVSGGVASFVCQATGDPKPRVTWNKKGKKVNSQRFETIDFDESSGAVLRIQPLRTPRDENVYECVAQNSVGEITIHAKLTVLREDQLPPGFPNIDMGPQLKVVERTRTATMLCAASGNPDPEITWFKDFLPVDPSASNGRIKQLRSGALQIESSEETDQGKYECVATNSAGVRYSSPANLYVRVRRVAPRFSILPMSHEIMPGGNVNITCVAVGSPMPYVKWMQGAEDLTPEDDMPVGRNVLELTDVKDSANYTCVAMSSLGVIEAVAQITVKSLPKAPGTPVVTENTATSITVTWDSGNPDPVSYYVIEYKSKSQDGPYQIKEDITTTRYSIGGLSPNSEYEIWVSAVNSIGQGPPSESVVTRTGEQAPASAPRNVQARMLSATTMIVQWEEPVEPNGLIRGYRVYYTMEPEHPVGNWQKHNVDDSLLTTVGSLLEDETYTVRVLAFTSVGDGPLSDPIQVKTQQGVPGQPMNLRAEAKSETSIGLSWSAPRQESVIKYELLFREGDRGREVGRTFDPTTAFVVEDLKPNTEYAFRLAARSPQGLGAFTAVVRQRTLQAKPSAPPQDVKCTSLRSTAILVSWRPPPPETHNGALVGYSVRYRPLGSEDPDPKEVNNIPPTTTQILLEALEKWTEYRVTAVAYTEVGPGPESSPVVVRTDEDVPSAPPRKVEAEALNATAIRVLWRSPTPGRQHGQIRGYQVHYVRMEGAEARGPPRIKDIMLADAQEMVITNLQPETAYSITVAAYTMKGDGARSKPKVVVTKGAVLGRPTLSVQQTPEGSLLARWEPPADAAEDPVLGYRLQFGREDAAPATLELAAWERRFAAPAHKGATYVFRLAARGRAGLGEEAAAALSIPEDAPRGFPQILGAAGNVSAGSVLLRWLPPVPAERNGAIIKYTVSVREAGAPGPATETELAAAAQPGAETALTLRGLRPETAYELRVRAHTRRGPGPFSPPLRYRLARDPVSPKNFKVKMIMKTSVLLSWEFPDNYNSPTPYKIQYNGLTLDVDGRTTKKLITHLKPHTFYNFVLTNRGSSLGGLQQTVTARTAFNMLSGKPSVAPKPDNDGFIVVYLPDGQSPVTVQNYFIVMVPLRKSRGGQFPVLLGSPEDMDLEELIQDISRLQRRSLRHSRQLEVPRPYIAARFSILPAVFHPGNQKQYGGFDNRGLEPGHRYVLFVLAVLQKNEPTFAASPFSDPFQLDNPDPQPIVDGEEGLIWVIGPVLAVVFIICIVIAILLYKNKPDSKRKDSEPRTKCLLNNADLAPHHPKDPVEMRRINFQTPGMLSHPPIPITDMAEHMERLKANDSLKLSQEYESIDPGQQFTWEHSNLEANKPKNRYANVIAYDHSRVILQPLEGIMGSDYINANYVDGYRRQNAYIATQGPLPETFGDFWRMVWEQRSATVVMMTRLEEKSRIKCDQYWPNRGTETYGFIQVTLLDTMELATFCVRTFSLHKNGSSEKREVRHFQFTAWPDHGVPEYPTPFLAFLRRVKTCNPPDAGPIVVHCSAGVGRTGCFIVIDAMLERIKTEKTVDVYGHVTLMRSQRNYMVQTEDQYGFIHEALLEAVGCGNTEVPARSLYTYIQKLAQVEPGEHVTGMELEFKRLASSKAHTSRFITASLPCNKFKNRLVNILPYESSRVCLQPIRGVEGSDYINASFIDGYRQQKAYIATQGPLAETTEDFWRALWENNSTIVVMLTKLREMGREKCHQYWPAERSARYQYFVVDPMAEYNMPQYILREFKVTDARDGQSRTVRQFQFTDWPEQGAPKSGEGFIDFIGQVHKTKEQFGQDGPISVHCSAGVGRTGVFITLSIVLERMRYEGVVDIFQTVKVLRTQRPAMVQTEDEYQFCFQAALEYLGSFDHYAT

SEQ ID NO: 5 - protein, 98 aa, Homo sapiens
EEPPRFIKEPKDQIGVSGGVASFVCQATGDPKPRVTWNKKGKKVNSQRFETIEFDESAGAVLRIQPLRTPRDENVYECVAQNSVGEITVHAKLTVLRE

SEQ ID NO: 6 - protein, 117 aa, Homo sapiens
DQLPSGFPNIDMGPQLKVVERTRTATMLCAASGNPDPEITWFKDFLPVDPSASNGRIKQLRSETFESTPIRGALQIESSEETDQGKYECVATNSAGVRYSSPANLYVRELREVRRVA

SEQ ID NO: 7 - protein, 90 aa, Homo sapiens; identical to SEQ ID NO: 3

SEQ ID NO: 8 - protein, 1948 aa, Homo sapiens
MAPTWGPGMVSVVGPMGLLVVLLVGGCAAEEPPRFIKEPKDQIGVSGGVASFVCQATGDPKPRVTWNKKGKKVNSQRFETIEFDESAGAVLRIQPLRTPRDENVYECVAQNSVGEITVHAKLTVLREDQLPSGFPNIDMGPQLKVVERTRTATMLCAASGNPDPEITWFKDFLPVDPSASNGRIKQLRSETFESTPIRGALQIESSEETDQGKYECVATNSAGVRYSSPANLYVRELREVRRVAPRFSILPMSHEIMPGGNVNITCVAVGSPMPYVKWMQGAEDLTPEDDMPVGRNVLELTDVKDSANYTCVAMSSLGVIEAVAQITVKSLPKAPGTPMVTENTATSITITWDSGNPDPVSYYVIEYKSKSQDGPYQIKEDITTTRYSIGGLSPNSEYEIWVSAVNSIGQGPPSESVVTRTGEQAPASAPRNVQARMLSATTMIVQWEEPVEPNGLIRGYRVYYTMEPEHPVGNWQKHNVDDSLLTTVGSLLEDETYTVRVLAFTSVGDGPLSDPIQVKTQQGVPGQPMNLRAEARSETSITLSWSPPRQESIIKYELLFREGDHGREVGRTFDPTTSYVVEDLKPNTEYAFRLAARSPQGLGAFTPVVRQRTLQSKPSAPPQDVKCVSVRSTAILVSWRPPPPETHNGALVGYSVRYRPLGSEDPEPKEVNGIPPTTTQILLEALEKWTQYRITTVAHTEVGPGPESSPVVVRTDEDVPSAPPRKVEAEALNATAIRVLWRSPAPGRQHGQIRGYQVHYVRMEGAEARGPPRIKDVMLADAQWETDDTAEYEMVITNLQPETAYSITVAAYTMKGDGARSKPKVVVTKGAVLGRPTLSVQQTPEGSLLARWEPPAGTAEDQVLGYRLQFGREDSTPLATLEFPPSEDRYTASGVHKGATYVFRLAARSRGGLGEEAAEVLSIPEDTPRGHPQILEAAGNASAGTVLLRWLPPVPAERNGAIVKYTVAVREAGALGPARETELPAAAEPGAENALTLQGLKPDTAYDLQVRAHTRRGPGPFSPPVRYRTFLRDQVSPKNFKVKMIMKTSVLLSWEFPDNYNSPTPYKIQYNGLTLDVDGRTTKKLITHLKPHTFYNFVLTNRGSSLGGLQQTVTAWTAFNLLNGKPSVAPKPDADGFIMVYLPDGQSPVPVQSYFIVMVPLRKSRGGQFLTPLGSPEDMDLEELIQDISRLQRRSLRHSRQLEVPRPYIAARFSVLPPTFHPGDQKQYGGFDNRGLEPGHRYVLFVLAVLQKSEPTFAASPFSDPFQLDNPDPQPIVDGEEGLIWVIGPVLAVVFIICIVIAILLYKNKPDSKRKDSEPRTKCLLNNADLAPHHPKDPVEMRRINFQTPDSGLRSPLREPGFHFESMLSHPPIPIADMAEHTERLKANDSLKLSQEYESIDPGQQFTWEHSNLEVNKPKNRYANVIAYDHSRVILQPIEGIMGSDYINANYVDGYRCQNAYIATQGPLPETFGDFWRMVWEQRSATIVMMTRLEEKSRIKCDQYWPNRGTETYGFIQVTLLDTIELATFCVRTFSLHKNGSSEKREVRQFQFTAWPDHGVPEYPTPFLAFLRRVKTCNPPDAGPIVVHCSAGVGRTGCFIVIDAMLERIKPEKTVDVYGHVTLMRSQRNYMVQTEDQYSFIHEALLEAVGCGNTEVPARSLYAYIQKLAQVEPGEHVTGMELEFKRLANSKAHTSRFISANLPCNKFKNRLVNIMPYESTRVCLQPIRGVEGSDYINASFIDGYRQQKAYIATQGPLAETTEDFWRMLWENNSTIVVMLTKLREMGREKCHQYWPAERSARYQYFVVDPMAEYNMPQYILREFKVTDARDGQSRTVRQFQFTDWPEQGVPKSGEGFIDFIGQVHKTKEQFGQDGPISVHCSAGVGRTGVFITLSIVLERMRYEGVVDIFQTVKMLRTQRPAMVQTEDEYQFCYQAALEYLGSFDHYAT

SEQ ID NO: 9 - protein, 1228 aa, Mus musculus
EEPPRFIREPKDQIGVSGGVASFVCQATGDPKPRVTWNKKGKKVNSQRFETIDFDESSGAVLRIQPLRTPRDENVYECVAQNSVGEITIHAKLTVLREDQLPPGFPNIDMGPQLKVVERTRTATMLCAASGNPDPEITWFKDFLPVDPSASNGRIKQLRSGALQIESSEETDQGKYECVATNSAGVRYSSPANLYVRVRRVAPRFSILPMSHEIMPGGNVNITCVAVGSPMPYVKWMQGAEDLTPEDDMPVGRNVLELTDVKDSANYTCVAMSSLGVIEAVAQITVKSLPKAPGTPVVTENTATSITVTWDSGNPDPVSYYVIEYKSKSQDGPYQIKEDITTTRYSIGGLSPNSEYEIWVSAVNSIGQGPPSESVVTRTGEQAPASAPRNVQARMLSATTMIVQWEEPVEPNGLIRGYRVYYTMEPEHPVGNWQKHNVDDSLLTTVGSLLEDETYTVRVLAFTSVGDGPLSDPIQVKTQQGVPGQPMNLRAEAKSETSIGLSWSAPRQESVIKYELLFREGDRGREVGRTFDPTTAFVVEDLKPNTEYAFRLAARSPQGLGAFTAVVRQRTLQAKPSAPPQDVKCTSLRSTAILVSWRPPPPETHNGALVGYSVRYRPLGSEDPDPKEVNNIPPTTTQILLEALEKWTEYRVTAVAYTEVGPGPESSPVVVRTDEDVPSAPPRKVEAEALNATAIRVLWRSPTPGRQHGQIRGYQVHYVRMEGAEARGPPRIKDIMLADAQEMVITNLQPETAYSITVAAYTMKGDGARSKPKVVVTKGAVLGRPTLSVQQTPEGSLLARWEPPADAAEDPVLGYRLQFGREDAAPATLELAAWERRFAAPAHKGATYVFRLAARGRAGLGEEAAAALSIPEDAPRGFPQILGAAGNVSAGSVLLRWLPPVPAERNGAIIKYTVSVREAGAPGPATETELAAAAQPGAETALTLRGLRPETAYELRVRAHTRRGPGPFSPPLRYRLARDPVSPKNFKVKMIMKTSVLLSWEFPDNYNSPTPYKIQYNGLTLDVDGRTTKKLITHLKPHTFYNFVLTNRGSSLGGLQQTVTARTAFNMLSGKPSVAPKPDNDGFIVVYLPDGQSPVTVQNYFIVMVPLRKSRGGQFPVLLGSPEDMDLEELIQDISRLQRRSLRHSRQLEVPRPYIAARFSILPAVFHPGNQKQYGGFDNRGLEPGHRYVLFVLAVLQKNEPTFAASPFSDPFQLDNPDPQPIVDGEEG

SEQ ID NO: 10 - protein, 1253 aa, Homo sapiens
EEPPRFIKEPKDQIGVSGGVASFVCQATGDPKPRVTWNKKGKKVNSQRFETIEFDESAGAVLRIQPLRTPRDENVYECVAQNSVGEITVHAKLTVLREDQLPSGFPNIDMGPQLKVVERTRTATMLCAASGNPDPEITWFKDFLPVDPSASNGRIKQLRSETFESTPIRGALQIESSEETDQGKYECVATNSAGVRYSSPANLYVRELREVRRVAPRFSILPMSHEIMPGGNVNITCVAVGSPMPYVKWMQGAEDLTPEDDMPVGRNVLELTDVKDSANYTCVAMSSLGVIEAVAQITVKSLPKAPGTPMVTENTATSITITWDSGNPDPVSYYVIEYKSKSQDGPYQIKEDITTTRYSIGGLSPNSEYEIWVSAVNSIGQGPPSESVVTRTGEQAPASAPRNVQARMLSATTMIVQWEEPVEPNGLIRGYRVYYTMEPEHPVGNWQKHNVDDSLLTTVGSLLEDETYTVRVLAFTSVGDGPLSDPIQVKTQQGVPGQPMNLRAEARSETSITLSWSPPRQESIIKYELLFREGDHGREVGRTFDPTTSYVVEDLKPNTEYAFRLAARSPQGLGAFTPVVRQRTLQSKPSAPPQDVKCVSVRSTAILVSWRPPPPETHNGALVGYSVRYRPLGSEDPEPKEVNGIPPTTTQILLEALEKWTQYRITTVAHTEVGPGPESSPVVVRTDEDVPSAPPRKVEAEALNATAIRVLWRSPAPGRQHGQIRGYQVHYVRMEGAEARGPPRIKDVMLADAQWETDDTAEYEMVITNLQPETAYSITVAAYTMKGDGARSKPKVVVTKGAVLGRPTLSVQQTPEGSLLARWEPPAGTAEDQVLGYRLQFGREDSTPLATLEFPPSEDRYTASGVHKGATYVFRLAARSRGGLGEEAAEVLSIPEDTPRGHPQILEAAGNASAGTVLLRWLPPVPAERNGAIVKYTVAVREAGALGPARETELPAAAEPGAENALTLQGLKPDTAYDLQVRAHTRRGPGPFSPPVRYRTFLRDQVSPKNFKVKMIMKTSVLLSWEFPDNYNSPTPYKIQYNGLTLDVDGRTTKKLITHLKPHTFYNFVLTNRGSSLGGLQQTVTAWTAFNLLNGKPSVAPKPDADGFIMVYLPDGQSPVPVQSYFIVMVPLRKSRGGQFLTPLGSPEDMDLEELIQDISRLQRRSLRHSRQLEVPRPYIAARFSVLPPTFHPGDQKQYGGFDNRGLEPGHRYVLFVLAVLQKSEPTFAASPFSDPFQLDNPDPQPIVDGEEG

SEQ ID NO: 11 - protein, 86 aa, Artificial Sequence
  Synthetic Construct
PKDQIGVSGGVASFVCQATGDPKPRVTWNKKGKKVNSQRFETIEFDESAGAVLRIQPLRTPRDENVYECVAQNSVGEITVHAKLTV

SEQ ID NO: 12 - protein, 82 aa, Artificial SEquence
  Synthetic Construct
ATMLCAASGNPDPEITWFKDFLPVDPSASNGRIKQLRSETFESTPIRGALQIESSEETDQGKYECVATNSAGVRYSSPANLY

SEQ ID NO: 13 - protein, 69 aa, Artificial Sequence
  Synthetic Construct
GGNVNITCVAVGSPMPYVKWMQGAEDLTPEDDMPVGRNVLELTDVKDSANYTCVAMSSLGVIEAVAQIT